(19) United States
(12) Patent Application Publication
(10) Pub. No.: US 2013/0261176 A1
(43) Pub. Date: Oct. 3, 2013

(54) Method of Detecting Mechanosensing Responses in Bone Cells

(71) Applicant: University Of Tennessee Research Foundation, Knoxville, TN (US)

(72) Inventors: LEIGH DARRYL QUARLES, MEMPHIS, TN (US); ZHOUSHENG XIAO, GERMANTOWN, TN (US)

(21) Appl. No.: 13/804,091

(22) Filed: Mar. 14, 2013

Related U.S. Application Data
(60) Provisional application No. 61/618,447, filed on Mar. 30, 2012.

(51) Int. Cl. C12Q 1/68; G01N 33/68

(52) U.S. Cl. CPC C12Q 1/6883; G01N 33/6887
USPC 514/468; 435/6.11; 435/7.1

(57) ABSTRACT

This invention relates to a method of detecting mechanosensing responses in bone. More specifically, the present invention provides a method to detect the expression levels of genes in mechanosensing complex to predict impaired mechanosensing response. Further, the present invention disclosed a therapeutic method for preventing bone loss and defective in bone formation by modulating the activity of the mechanosensing complex.

### RELATED APPLICATIONS

The presently disclosed invention claims the benefit of U.S. provisional patent application Ser. No. 61/618,447 filed Mar. 30, 2012 under 35 USC Sec 119 (e) (the disclosure of which is incorporated herein by reference in its entirety).

### GOVERNMENT INTEREST

This invention was developed under grant R01-DK083303 and R21-AR056794 from the National Institutes of Health, the United States may retain some rights to this invention.

### FIELD OF THE INVENTION

This invention relates to a method of detecting mechanosensing responses in bone. More specifically, the present invention provides a method to detect the expression levels of genes in mechanosensing complex to predict impaired mechanosensing response. Further, the present invention disclosed a therapeutic method for preventing bone loss and defective in bone formation by modulating the activity of the mechanosensing complex.

### BACKGROUND OF THE INVENTION

Mechanosensing is the responsivity to mechanical stimuli, especially at the cellular level or below. Various factors are involved in mechanosensing responses. For example, osteocytes, which are embedded in bone matrix and have a high degree of interconnectivity, are postulated to be an important mechanosensing cell in bone. The central role of osteocytes in bone metabolism is supported by the development of osteoporosis and defective mechanosensation after the ablation of osteocytes, as well as by the fact that osteocytes secrete paracrine and systemic regulators affecting osteoblast-mediated bone formation and resorption, systemic phosphate and vitamin D homeostasis, and bone mineralization, such as sclerostin (Sost) dickkopf-1 (Dkk1), fibroblast growth factor 23 (FGF23), and matrix extracellular phosphoglycoproteins (MEPE), respectively. Mature osteoblasts and chondrocytes also detect and respond to mechanical loading.

Many cell surface molecules (e.g., Lrp5 and ERα) and intracellular signaling pathways have been postulated to transduce the mechanosensing response in osteoblasts and osteocytes. (Armstrong, V. J et al. (2007) Wnt/beta-catenin signaling is a component of osteoblastic bone cell early responses to load-bearing and requires estrogen receptor alpha, J. Biol. Chem. 282, 20715-20727) Putative mechanosensing molecules include several G-protein-coupled receptors (e.g., prostaglandin and purinergic receptors), integrin receptors, connexins/gap-junctions, hemichannels, and stretch-activated ion channels. Although the mechanosensing function of many of these pathways has been demonstrated in cell culture models, their mechanosensation in vivo remains to be established. In addition, multiple autocrine and paracrine factors and signaling pathways have been implicated, including prostaglandins (e.g., PGE2), nitric oxide (NO), ATP, growth factors (e.g., TGF-β, FGF2, and IGFs), and Wnt/β-catenin signaling; (Case, N., et al. (2008) Beta-catenin levels influence rapid mechanical responses in osteoblasts. J. Biol. Chem. 283, 29196-29205) however, the molecular identity of the environmental stimuli or relevant intracellular signaling pathways involved in mechanosensing remains uncertain.

Polycystin-1 (PC1 or Pkd1) has been reported as part of a mechanosensing complex in renal epithelial cells and is highly expressed in osteoblasts and osteocytes and plays an important role in both skeletal development and postnatal bone homeostasis through intracellular calcium and Runx2-dependent signaling mechanisms. (Xiao, Z., et al. (2006) Cilia-like structures and polycystin-1 in osteoblasts/osteocytes and associated abnormalities in skeletogenesis and Runx2 expression. J. Biol. Chem. 281, 30884-30895). The function of Pkd1 as a mechanosensor in bone, however, has not been established.

Additionally, primary cilia house many signaling pathways that may be involved in osteoblasts development and postnatal osteoblast functions including polycystin-1 (Pkd1) and 2 (PC2 or Pkd2), which form a mechanosensing signaling complex that colocalizes to primary cilia. The C-terminal region of Kif3a has recently been shown to bind to the C-terminus of PC2, resulting in its localization and function in primary cilium. Other studies have reported that Kif3b, the other motor subunit of kinesin-2, serves as a linker between PC2 and fibrocystin/polyductin, the gene product of PKHD1, the gene responsible for autosomal recessive PKD (ARPKD). Polycystins and primary cilia have interdependent function in renal epithelial cells (Nauli, S. M., et al. (2003) Polycystins 1 and 2 mediate mechanosensation in the primary cilium of kidney cells. Nat. Genet. 33, 129-137) mutations of polycystins and genes required for ciliogenesis lead to a common polycystic kidney disease phenotype.

The skeleton adapts to alterations in mechanical loading by changing bone structure. Increased load results in increased bone mass as a result of increased osteoblast-mediated bone formation, whereas skeletal unloading, which occurs with immobilization, disuse, and exposure to low gravity, leads to low bone mass from increased bone resorption. The cellular targets in bone and the molecular mechanisms that sense changes in mechanical load however are uncertain. Increased mechanical load increases bone mass via stimulation of osteoblast-mediated bone formation, whereas skeletal unloading, leads to low bone mass from increased bone resorption. Mechanical forces (loading and unloading) are important regulators of bone mass. Alterations in skeletal loading have profound biological and clinical effects on bone remodeling. Disuse and/or weightlessness leads to osteoporosis while exercise and/or increase in muscle strength increases bone mass.

To fully understand the mechanosensing response requires a detailed understanding of 1) the mechanical forces in bone, 2) the principal mechanosensing cells in bone, 3) the molecular identity of the mechanosensor, 4) the biologically relevant intracellular and paracrine signaling pathways, 5) the effectors (i.e., gene products altering osteoblasts and osteoclasts-mediated bone remodeling). Elucidating this multi-step cascade is fundamental to the understanding of basic bone physiology and will provide a basis to develop treatments to increase bone mass and/or prevent bone loss in unloading conditions, such as immobilization and space flight.

The central role of osteocytes in bone metabolism is supported by the development of osteoporosis and defective mechanosensation after the ablation of osteocytes, as well as by the fact that osteocytes secrete paracrine and systemic regulators affecting osteoblast-mediated bone formation and resorption There is a growing consensus that osteocytes in bone, which form a 3-D interconnected network that makes them ideal for a mechanosensing role and regulate both osteoblast and osteoclast activities in response to mechanical loading and unloading. However, the primary/proximate molecular entity responsible for sensing and transducing the mechanosensing response in osteocytes remains uncertain. However, the molecular mechanism(s) whereby osteocytes sense and transduce mechanical signals into anabolic responses in bone remains uncertain.

There are many competing ideas regarding the components of the multi-step molecular mechanosensing cascade in bone. Many cell surface molecules (e.g., Lrp5 and membrane forms of ERα) and intracellular and paracrine signaling pathways have been postulated to be involved in the mechanical loading response in osteocytes and osteoblasts. But most these molecules do not have the structural properties to sense mechanical forces including fluid flow, and likely down-stream of a primary molecular sensor). Other putative mechanosensing molecules via both fluid flow and stretch-activated pathways have also been implicated. Although the ability of these molecules to modify mechanosensing responses has been demonstrated in cell culture models in vitro, confirmation of their mechanosensing properties in vivo and a conceptual/structural frame work for how these molecules sense forces have not been established. Therefore, it is desirable to study the biologically relevance of primary cilia and polycystins as mechanosensor in osteocytes.

### SUMMARY OF THE INVENTION

The present invention provides in some embodiments a method for detecting impaired the mechanosensing function in bone in a subject by detecting the amount of one or more biomarker(s) in the mechanosensing complex in a biological sample of a subject and calculating the amount of the one or more biomarker(s) in the sample and comparing to a control level of the biomarkers, and a measurable difference in the amount of the one or more biomarker(s) in the sample as compared to the control level indicates a greater likelihood that the subject suffers from impaired mechanosensing function. The present invention further provides in some embodiments, a method of diagnosing impaired bone conditions in a subject by determining a measurable change in the amount of at least one biomarker.

The one or more biomarkers in the present invention can be biomarkers in mechanosensing complex which are made of polycystin-complex and primary cilia in bone and other cells. In one embodiment, the biomarkers in the polycystin-complex are made of Pkd1 and Pkd2. In another embodiment, the biomarker in primary cilia comprises Kif3a. In some embodiments, methods for determining an amount in the sample of biomarkers are disclosed. The biological sample in the present invention is made of blood, serum, plasma, bone tissues, femur tissues, osteoblast cells. In some embodiments, the subject is a mammal. In a particular embodiment, the subject is mice.

In the present invention, the method to determine the amount of the at least one biomarkers involves one or more techniques known to an ordinary skill in the art, including mRNA and protein measuring assays. Exemplary RNA measuring assays include real time RT-PCR (reverse transcription polymerase chain reaction) and the exemplary protein measuring assays include western blot analysis and immunoassay analysis.

In the present invention, the impaired bone conditions can be, but not limited to the following: reduction in bone marrow density, bone volume and cortical thickness, and mineral apposition rate (MAR); reductions in osteoblast and osteoclastic markers; diminished osteoblast-mediated bone formation and osteoclast-mediated bone resorption; low-turnover osteopenia; postnatal bone formation; bone mass, structure, geometry, and mechanical properties; impaired osteoblastic differentiation and maturation; reduction in length of primary cilia; flow-induced intracellular calcium concentration; attenuated mechanoresponsive gene expression; impaired Hh signaling in osteoblasts; attenuated Wnt/β-catenin signaling in osteoblasts.

In another embodiment of the present invention a kit to detect impaired mechanosensing function in bone is provided. This kit includes a solid support having first agent to bind polycystin-complex and a second agent to identify the bond complex. In another embodiment, the first agent is a molecular probe and the second agent is a molecular primer. Yet in another preferred embodiment, the kit includes a first antibody that can bind to and detect the amount of one of the biomarker proteins in the mechanosensing complex, and, optionally, a different antibody that is capable of bind to the first antibody or the biomarker protein for detecting the amount of the biomarker protein.

In some embodiments, the kit may include an agent to bind proteins or nucleic acids and means to determine the protein and nucleic acids levels of proteins or mRNAs in a biological sample. In one preferred embodiment, the kit includes forward and reverse primers capable of amplifying at least one biomarker in the mechanosensing complex in a biological sample from a subject.

The current invention further relates to a therapeutic method for preventing bone loss and defective in bone formation by modulating the activity of the mechanosensing complex. More particularly, the mechanosensing complex is made of primary cilia and polycystins which represent the biologically relevant mechanosensor in osteocytes. In a preferred embodiment, the method involves contacting the mechanosensing complex with a ligand which binds to the polypeptide of these genes in mechanosensing complex in a sufficient concentration to modulate the mechanosensing activity of the mechanosensing complex.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 shows Dmp1-Cre-mediated conditional deletion of Pkd1 from the floxed Pkd1 allele (Pkd1^{flox}) in different tissues.

FIG. 2 illustrates Oc-Cre-mediated conditional deletion of Kif3a from the floxed Kif3a allele (Kif3a^{flox}) in different tissues.

FIG. 3 illustrates Dmp1-Cre-mediated somatic loss of Pkd1 leads to osteopenia.

FIG. 4 is Oc-Cre-mediated somatic deletion under Kif3a-deficient background leads to osteopenia.

FIG. 5 shows age-dependent effects of Dmp1-Cre-mediated Pkd1^{Δflox} allele on bone mass.

FIG. 6 is age-dependent effects of global and/or Oc-Cre-mediated conditional deletion of Kif3a on bone mass and structure.

FIG. 7 shows effects of Dmp1-Cre-mediated Pkd1 deletion on osteoblastic proliferation and maturation ex vivo.

FIG. 8 is effects of global and/or Oc-Cre-mediated conditional deletion of Kif3a on osteoblastic proliferation and maturation, as well as gene expression profiles ex vivo.

FIG. 9 shows effects of Pkd1 deletion and mutation on baseline and flow-induced intracellular calcium ([Ca²⁺]ᵢ) response in osteoblasts.

FIG. 10 is effects of global and/or Oc-Cre-mediated conditional deletion of Kif3a on baseline and flow-induced intracellular calcium ([Ca²]ᵢ) response, as well as mechanoresponsive gene expression in osteoblasts.

FIG. 11 shows conditional deletion and mutation of Pkd1 in osteocytes impairs anabolic response to mechanical loading.

FIG. 12 is effects of global and/or Oc-Cre-mediated conditional deletion of Kif3a on Hh and Wnt signaling in bone and osteoblasts.

FIG. 13 is the effects of conditional deletion of Pkd1 in bone on fracture healing.

### DETAILED DESCRIPTION OF THE INVENTION

The present invention may be understood more readily by reference to the following detailed description of the invention. It is to be understood that this invention is not limited to the specific devices, methods, conditions or parameters described herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

In the context of the methods of this invention, biomarkers can include molecular cellular markers that indicate the biologically relevant mechanosensor in osteocytes. Osteocytes are a bone cell, formed when an osteoblast becomes embedded in the matrix it has secreted. Mechanosensors are defined as responsivity to mechanical stimuli, especially at the cellular level or below.

In a preferred embodiment, the biomarker is a characteristic morphological pattern produced in osteocytes, but can include individual marker protein, or its encoding mRNA. The biomarker gene refers to a DNA, mRNA or cDNA transcripts, and/or coding sequence and related polynucleotides or oligonucleotides that are complementary to a nucleic acid sequence or a part thereof, and polynucleotides or oligonucleotides that can be hybridized or bind to a nucleic acid sequence.

These biomarkers are characterized by measurement of a molecule that can provide information as to the state of bone function of a subject, and any biomarker for this function is contemplated by this invention. In various exemplary embodiments, measurements of at least of one of the biomarkers are used alone or combined with other data obtained to determine the state of impaired bone function of the subject. The biomarkers used herein refer to proteins nucleic acids. The term “protein” includes peptide, polypeptide or oligopeptide which is two or more amino acids linked by one or more peptide bonds. In some embodiment, the biomarker is a nucleic acid. The term “nucleic acid” or “oligonucleotide” disclosed herein refers to at least two nucleotides covalently linked together.

Detection of the biomarker can be accomplished by a variety of detection methodologies including for example affinity capture followed by mass spectrometry, or a traditional immunoassay. One skilled in the art can prepare bio-specific capture reagents, such as antibodies, to capture the analysts. Antibodies can be produced by immunizing animals with the bio-molecules. This invention contemplates traditional immunoassays including, for example, sandwich immunoassays including ELISA or fluorescence-based immunoassays, as well as other enzyme immunoassays. In another aspect this invention provides a composition comprising a bio-specific capture reagent, such as an antibody, bound to a biomarker of this invention. For example, an antibody that is directed against a biomarker of this invention and that is bound to the biomarker, is useful for detecting the biomarker. In one embodiment, the biospecific capture reagent is bound to a solid support, such as a bead, a chip, a membrane or a microtiter plate. In one embodiment, the kit comprises a solid support, such as a chip, a microtiter plate or a bead or resin having an adsorbent attached thereon, wherein the adsorbent binds a biomarker of the invention.

In one embodiment, the at least one biomarker is measured by capturing the biomarker on an adsorbent of a SELDI probe and detecting the captured biomarkers by laser desorption-ionization mass spectrometry.

In one embodiment of the present invention, a kit to detect impaired mechanosensing function in bone is provided. The biomarkers can be bound to a biochip array. More specifically the biomarkers in mechanosensing complex are made of a polycystin-complex and primary cilia. The polycystin-complex is made of Pkd1 and Pkd2 while the primary cilium is Kif3a. This kit includes a solid support having first agent to bind polycystin-complex and a second agent to identify the bond complex. In one embodiment, the first agent is a molecular probe or primer and in the second agent is a molecular probe or primer. (Sato K, et. al, Microchip-based immunoassay system with branching multichannel for simultaneous determination of interferon-gamma, Electrophoresis, 2002 mar; 23 (5):734-9)). The kit can include an instructional manual, calibration software, washing solutions and sampling tool such as pipettes as known to one skilled in the art.

In another embodiment of the present invention, kit that can detect the mRNA levels of the biomarkers may include an agent to bind proteins or nucleic acids and means to determine the protein and nucleic acids levels of proteins or mRNAs in a biological sample. In one preferred embodiment, the kit includes forward and reverse primers capable of amplifying at least one biomarker in the mechanosensing complex in a biological sample from a subject.

In one embodiment, quantitative RT-PCR is used to determine expression levels of at least one of the biomarkers. A preferred embodiment provides a real-time quantitative RT-PCR method for detecting at least one biomarkers in the mechanosensing complex in a biological sample. (see Chen, C. et. al, Real-time quantification of microRNAs by stem-loop RT-PCR, Nucleic Acids Res. 2005; 33(20): e179.)

In some embodiments, quantitative RT-PCR uses a forward and a reverse primer for each biomarker gene of the mechanosensing complex. Primers having sequence identical to SEQ ID NO: 1-8 and SEQ ID NO: 47-52 are the forward and reverse primers as shown in Example 1 and 2 are used for quantitative RT-PCR.

In another embodiment, this invention provides methods for determining the therapeutic efficacy of a pharmaceutical drug, for treating bone loss. These methods are useful in performing clinical trials of the drug, as well as monitoring the progress of a patient on the drug therapy or clinical trials involve administering the drug for a particular regimen.

In one embodiment, the present invention provides that conditional Pkd1 heterozygotes (Dmp1-Cre; Pkd^{1flox/+}) and null mice (Pkd^{1Dmp1-cKO}) exhibited a ˜40% and ˜90% decrease, respectively, in functional Pkd1 transcripts in bone. Femoral bone mineral density (12% vs. 27%), trabecular bone volume (32% vs. 48%), and cortical thickness (6% vs. 17%) were reduced proportionate to the reduction of Pkd1 gene dose as were mineral apposition rate (MAR) and expression of Runx2-II, Osteocalcin, Dmp1, and Phex. Anabolic load-induced periosteal lamellar MAR (0.58±0.14; Pkd^{1Dmp-cKO} vs. 1.68±0.34 μm/day; control) and increases in Cox-2, c-Jun, Wnt10b, Axin2, and Runx2-II gene expression were significantly attenuated in Pkd^{1Dpm1-cKO} mice compared to controls. Application of fluid shear stress to immortalized osteoblasts from Pkd^{1null/null} and Pkd^{1m1Bei/m1Bei}-derived osteoblasts failed to elicit the increments in cytosolic calcium observed in wild type controls. These data indicate that polycystin-1 is essential for the anabolic response to skeletal loading in osteoblasts/osteocytes.

Additionally, the present invention has shown that PC1 and PC2 form a polycystin-complex, which is co-localized to plasma membrane and primary cilia in bone and other cells. PC1/PC2 signaling is coupled to multiple intracellular signal pathways and cellular responses including increments in intracellular calcium, Calcineurin/NFAT, PI3K/Akt/GSK3β, G-proteins, Wnt/β-catenin, AP-1 activations, and sclerostin (SOST) secretion which have been linked to mechanosensing responses in osteocytes in bone. It was also found that PC1 and Kif3a signaling counterbalanced osteogenesis and adipogenesis through differential regulation of Runx2 and PPARγ expression in bone cells. Finally, is has been shown in this invention that Pkd1^{Dmp1-cKO} null mice had lower osteoclastgenesis/bone resorption and a resistance to tail suspension unloading-mediated bone loss, and siRNA knockdown of Pkd1 in MLO-Y4 osteocytes resulted in loss of flow-induced increase in [Ca2+]i. Together, these evidence has firmly established that osteocyte cilium (Kif3a) and the polycystin-complex composed of Pkd1 and Pkd2 forms a mechanosensor.

In anther embodiment, the present invention has investigated whether Kif3a in osteoblasts has a direct role in regulating postnatal bone formation. Kif3a was conditionally deleted in osteoblasts by crossing Osteocalcin (Oc)-Cre with Kif3a^{flox/null} mice. Conditional Kif3a null mice (Kif3a^{Oc-cKO}) had a 75% reduction in Kif3a transcripts in bone and osteoblasts. Conditional deletion of Kif3a resulted in the reduction of primary cilia number by 51% and length by 27% in osteoblasts. Kif3a^{Oc-cKO} developed osteopenia by 6 weeks-of-age compared with Kif3a^{flox/+} control mice, as evidenced by reductions in femoral bone mineral density (22%); trabecular bone volume (42%); and cortical thickness (17%). In contrast, Oc-Cre; Kif3a^{flox/+} and Kif3a^{flox/null} heterozygous mice exhibited no skeletal abnormalities. Loss of bone mass in Kif3a^{Oc-cKO} mice was associated with impaired osteoblast function in vivo, as reflected by a 54% reduction in mineral apposition rate and decreased expression of Runx2, Osterix, Osteocalcin, and Dmp1 compared to controls. Immortalized osteoblasts from Kif3a^{Oc-cKO} mice exhibited increased cell proliferation, impaired osteoblastic differentiation, and enhanced adipogenesis in vitro. Osteoblasts derived from Kif3a^{Oc-cKO} mice also had lower basal cytosolic calcium levels and impaired intracellular calcium responses to fluid flow shear stress. Sonic hedgehog-mediated Gli2 expression and Wnt3a-mediated β-catenin/Axin2 expression were also attenuated in Kif3a^{Oc-cKO} bone and osteoblast cultures. These data indicate that selective deletion of Kif3a in osteoblasts disrupts primary cilia formation/function and impairs osteoblast-mediated bone formation through multiple pathways including intracellular calcium, hedgehog, and Wnt signaling.

Treatments to increase bone mass and/or prevent bone loss in unloading conditions, such as immobilization and space flight are contemplated by this invention. Compounds capable of modulating the activity of any of the biomarkers may be administered to subjects who are in need thereof of this therapy. For example, magnetic iron oxide nanoparticles have emerged as a clinical tool for biomedical applications in vitro and in vivo. These magnetic nanoparticles can provide targeted cell therapy because of their long blood retention time, biodegradability and low toxicity. These iron oxide nanoparticles have a large surface area and can be conjugated to a large number of disease-targeting ligands such as monoclonal antibodies that selectively bind to a targeting antigen, peptides, or small molecules for delivery of therapeutic agents. At the present time, a known ligand that can bind to Pkd1 is an antibody. An antibody specific to Pkd1 can be engineered by on skilled in the art to be linked to iron oxide nanoparticles. This iron oxide nanoparticle conjugated Pkd1 antibody can activate the mechanosensing response in response to a magnetic force. Additionally, Triptolide, a Chinese herb extract, has been demonstrated to bind and stimulate Pkd2 calcium channel activity in renal epithelial cells and markedly reduce the cysts formation in mouse ADPKD kidney. Moreover, novel Pkd1/Pkd2 ligands can be screened using the complex and a screening assay to screen libraries in a process known to on skilled in the relevant art.

The present invention conditionally deleted Pkd1 in mature osteoblasts/osteocytes by crossing Dmp1-Cre with Pkd^{1flox/m1Bei} mice in which the m1Bei allele is nonfunctional. The response to mechanical loading, in wild type and Pkd1-deficient mice was studied in vivo by ulna loading and ex vivo by measuring the response of isolated osteoblasts to fluid shear stress. The current invention characterized four genotypes, including conditional Dmp1-Cre; Pkd^{1flox/m1Bei} null mice (hereafter designated Pkd^{1Dmp1-cKO}), single conditional Dmp1-Cre; Pkd^{1flow/+} heterozygous mice, single Pkd^{1flox/m1Bei} heterozygous mice, and control Pkd^{1flox/+} mice. These mice were born at the expected Mendelian frequency, and Pkd^{1Dmp1-cKO} Dmp1-Cre; Pkd^{1flow/+} and Pkd^{1flox/m1Bei} exhibited survival indistinguishable from control mice. The normal survival of Pkd^{1Dmp1-cKO} mice contrasted with the perinatal lethality of homozygous Pkd^{1m1Bei/m1Bei} mice.

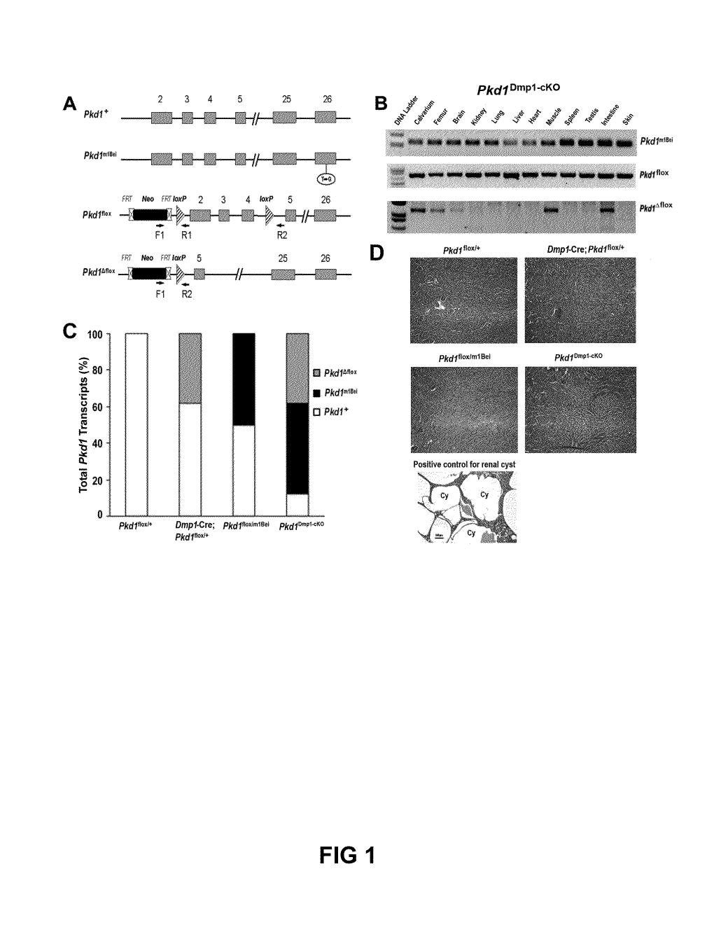

Now referring to FIG. 1 which illustrates Dmp1-Cre-mediated conditional deletion of Pkd1 from the floxed Pkd1 allele (Pkd1^{flox}) in different tissues. (A) Schematic illustration of wild type (Pkd1⁺), mutant (Pkd1^{m1Bei}), and floxed Pkd1 allele before (Pkd1^{flox}) and after deletion (Pkd1^{Δflox}) of the lox P cassette containing exon 2-4 via Cre-mediated recombination. “//” stands for all the introns and exons omitted between exon 5 and exon 25. (B) Genotype PCR analysis of different tissues that were harvested from 16-week-old Dmp1-Cre; Pkd1^{flox/m1Bei} mice. Pkd1 beier and floxed alleles existed in all tested tissues including bone. However, Dmp1-Cre-mediated recombination of excised floxed Pkd1 (Pkd1^{Δflox}) allele occurred in bone tissues such as calvarias and femur but also had a leakage in the brain, muscle, and intestine. (C) Real-time RT-PCR analysis of total Pkd1 transcripts. Expression of total Pkd1 transcripts was performed using Pkd1-allele-specific primers as described in Experimental Procedures. The normal Pkd1⁺ vs. cyclophilin A is normalized to the mean ratio of five control mice, which has been set to 1. The percentage of conditional deleted and mutant transcripts was calculated from the relative levels of the normal Pkd1⁺ transcripts in different Pkd1 exons. Data are expressed as the percentage of wild type (Pkd1 and Pkd1^{flox}), mutant (Pkd1^{m1Bei}), and conditional deleted (Pkd1^{Δflox}) Pkd1 allele expressions in Pkd1^{flox} control and Dmp1-Cre; Pkd1^{flox/-} conditional null mice. (D) Histology of adult kidney. Hematoxylin-eosin (H&E) stained sections from 6-week-old mice failed to identify any cystic tubules in either cortical or medullary regions of kidney from Dmp1-Cre; Pkd1^{flox/+} or Pkd1^{Dmp1-cKO} mice, consistent with the absence of Dmp1-Cre expression in the kidney. In contrast, ablation of Pkd1 in the kidney of 6-week old Col1a1(3.6)-Cre; Pkd1^{flox/flox} caused massive cyst formation, which served as a positive control. Cy, cyst. Scale bars, 100 μm.

To establish the conditional loss of Pkd1 from bone, PCR analysis by using a combination of primers that specifically detected floxed Pkd1 alleles (Pkd1^{flox}) and the excised floxed Pkd1 alleles (Pkd1^{Δflox}) in Dmp1-Cre; Pkd1^{flox/m1Bei} mice (FIG. 1A) was performed. It was found that Dmp1-Cre-mediated excision of the floxed Pkd1 (Pkd1^{Δflox}) allele occurred in bone tissues, including calvaria and femur, but also occurred in brain, muscle, and intestine, indicating that the Dmp1-Cre promoter is not specific for bone (FIG. 1B). No evidence for the Pkd1^{Δflox} allele in other tissues was found. Both the floxed Pkd1^{flox} and mutant Pkd1^{m1Bei} alleles were detected in all tested tissues (FIG. 1B).

To quantify the effect of combined use of floxed Pkd^{1flox} allele with functional Pkd1^{m1Bei} null allele to increase the net efficiency of Pkd1 inactivation by Cre-recombinase, we examined the percentage of Pkd1 conditional deleted and Pkd1^{m1Bei} mutant allele expressions in bone tissues from 6-week-old mice by real-time RT-PCR. As expected, both Pkd1^{flox/m1Bei} and Dmp1-Cre; Pkd1^{flox/m1Bei} mice expressed 50% Pkd1^{m1Bei} mutant (functional null) allele, whereas Dmp1-Cre; Pkd1^{flox/+} and Dmp1-Cre; Pkd1^{flox/m1Bei} mice exhibited approximately 40% excision of the floxed exon 2-4 from Pkd1, likely reflecting the expression of Dmp1-Cre mature osteoblasts just entering the osteoid, osteoid osteocytes, and mature osteocytes (61). The combined effect of Pkd1^{m1Bei} and Pkd1^{Δflox} in Dmp1-Cre; Pkd1^{flox/m1Bei} resulted in a net reduction of Pkd1 expression by ˜90% in bone (FIG. 1C). However, Dmp1-Cre-mediated conditional deletion of Pkd1 did not alter the appearance of primary cilia in cultured osteoblasts (data not shown). In addition, real-time RT-PCR to assess the expression level of the residual functional Pkd1 transcript confirmed the progressive reduction of Pkd1 message in conditional mutant mice, i.e., Pkd1^{flox/+} (100%), Dmp1-Cre; Pkd1^{flox/+} (60%), Pkd1^{flox/m1Bei} (50%), and Pkd1^{Dmp1-cKO} (10%) mice (data not shown). Consistent with the lack of Cre expression in the kidney, Pkd1^{Dmp1-cKO}, Dmp1-Cre; Pkd1^{flox/+} mice and mutant Pkd1^{m1Bei} mice demonstrated no cyst formation in the kidney, whereas use of a bone-restricted promoter Cre with kidney expression (Col1a3.6-Cre) resulted in the development of polycystic kidney disease in 6-week-old Col1a3.6-Cre; Pkd1^{flox/flox} mice (FIG. 1D).

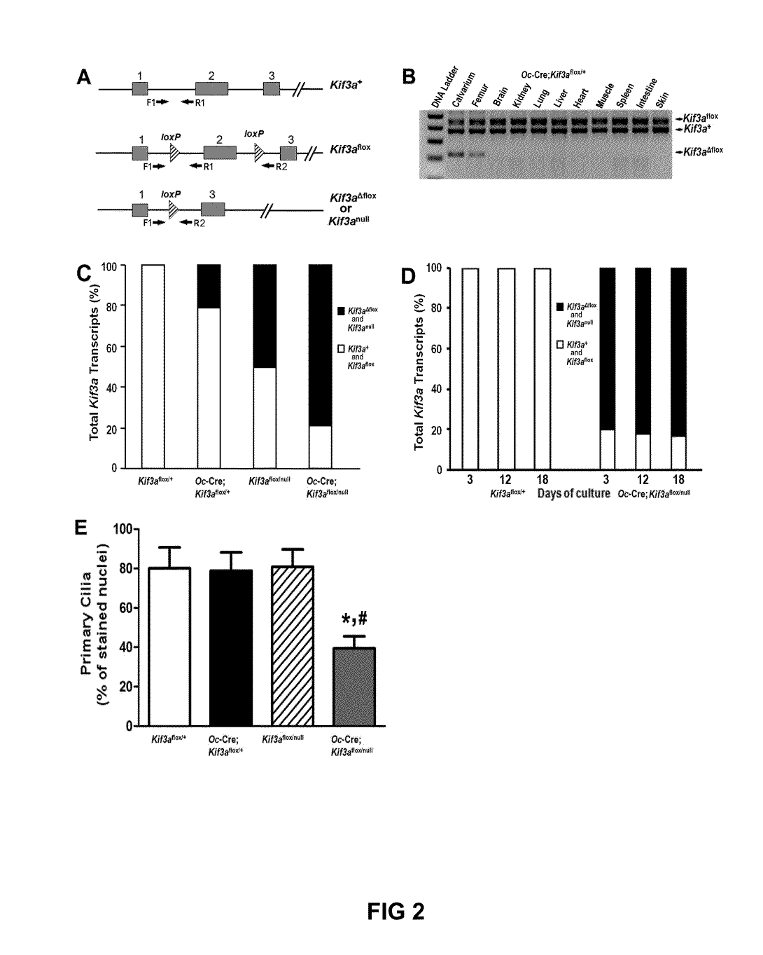

The four genotypes from the breeding strategy (Oc-Cre; Kif3a^{flox/null} or Kif3a^{Oc-cKO}, Oc-Cre; Kif3a^{flow/+}, Kif3a^{flox/null}, and Kif3a^{flox/+}) were born at the expected Mendelian frequency, and all Kif3a-deficient mice exhibited survival indistinguishable from control mice (Kif3a^{flox/+}, equivalent to wild type). The normal survival of conditional Kif3a^{Oc-cKO} null mice (Oc-Cre; Kif3a^{flox/null}) contrasts with the perinatal lethality of homozygous Kif3a^{null/null} mice. To confirm that the Kif3a-floxed allele was selectively deleted in bone, we performed PCR analysis by using a combination of primers that specifically detect floxed Kif3a alleles (Kif3a^{flox}) and the excised floxed Kif3a alleles (Kif3a^{Δflox}), as well as wild type alleles (Kif3a⁺) in Oc-Cre; Kif3a^{flox/+} mice (FIG. 2A). Oc-Cre expression is limited to mature osteoblasts (bone surface osteoblasts that synthesize new bone and osteocytes embedded in bone that regulate bone remodeling) with onset of expression just before birth (embryonic day 18.5, E18.5).

The present invention demonstrates that Oc-Cre-mediated floxed recombination occurred exclusively in tissues that contain osteoblasts, whereas nonskeletal tissues retained the intact floxed Kif3a alleles (Kif3a^{flox}) (FIG. 2B). Consistent with the lack of Cre expression in the kidney, Kif3a^{Oc-cKO}, Oc-Cre; Kif3a^{flox/+}, and Kif3a^{flox/null} mice demonstrated no cyst formation in the kidney (data not shown).

To quantify the effect of combined use of floxed Kif3a^{flux} allele with the nonfunctional Kif3a^{null} null allele to increase the net efficiency of Kif3a inactivation by Cre-recombinase, the percentage of Kif3a, conditional deleted (Kif3a^{Δflox}), and null (Kif3a^{null}) allele expressions in bone tissues from 6-week-old mice by real-time RT-PCR as examined. As expected, Kif3a^{flox/null} mice expressed 50% of Kif3a^{null} null allele, whereas Oc-Cre; Kif3a^{flox/+} mice exhibited approximately 25% excision of the floxed exon 2 from Kif3a, indicating that Oc-Cre mediated bone-specific deletion of the floxed Kif3a allele was incomplete (FIG. 2C). The combined effect of Kif3a^{Δflox} and Kif3a^{null} alleles in Oc-Cre; Kif3a^{flox/null} mice resulted in a net reduction of Kif3a expression by ˜75% in bone (FIG. 2C). Unlike a time-dependent increase of total Pkd1 transcripts during osteogenic culture, it was observed that no further increase of total Kif3a transcripts in control Kif3a^{flox/+} osteoblasts. Consistent with the reduction of Kif3a expression in bone, conditional Kif3a^{Oc-cKO} null osteoblasts showed more than 75% inactivation of Kif3a transcripts by real-time RT-PCR during 18 days of osteogenic culture (FIG. 2D). In addition, whereas both Oc-Cre; Kif3a^{flox/+} and Kif3a^{flow/null} heterozygous mice did not alter the appearance of primary cilia, conditional Kif3a^{Oc-cKO} null mice exhibited a marked reduction of primary cilia number in osteoblast cultures (FIG. 2E), in association with a 75% reduction in Kif3a transcript expression in osteoblasts derived from conditional Kif3a^{Oc-cKO} null mice (FIG. 2D).

Now referring to FIG. 2. Oc-Cre-mediated conditional deletion of Kif3a from the floxed Kif3a allele (Kif3a^{flox}) in different tissues. (A) Schematic illustration of wild type (Kif3a⁺), mutant (Kif3a^{Δflox} or Pkd1^{null}), and floxed Kif3a allele before (Kif3a^{flox}) and after deletion (Kif3a^{Δflox}) of the lox P cassette containing exon 2 via Cre-mediated recombination. “//” stands for all the introns and exons omitted after exon 3. (B) Genotyping PCR analysis of different tissues that were harvested from 6-week-old Oc-Cre; Kif3a^{flox/+} mice showed bone-specific deletion of the Kif3a gene. Oc-Cre-mediated recombination of excised floxed Kif3a (Kif3a^{Δflox}) allele occurred exclusively in bone, whereas nonskeletal tissues retained the floxed Kif3a allele (Kif3a^{flox}). (C-D) Real-time RT-PCR analysis of total Kif3a transcripts in bone and cultured osteoblasts. Expression of total Kif3a transcripts was performed using Kif3a-allele-specific primers as described in Materials and Methods. Data are expressed as the percentage of normal (wild type Kif3a⁺ and Kif3a^{flox}) and mutant (Kif3a^{Δflox} and Kif3a^{null}) Kif3a allele expressions in Kif3a^{flox/+} control, single Oc-Cre; Kif3a^{flox/+} and Kif3a^{flox/null} heterozygous mice, and conditional Kif3a^{Oc-cKO}-null mice. (E-F) Immunofluorescence of primary cilia in immortalized osteoblasts. Immunostaining of primary cilia (red) was performed with acetylated α-tubulin antibody as described in Materials and Methods. Counterstaining with a nuclear marker (DAPI blue) was used to calculate the percentage presence of primary cilia in immortalized osteoblasts. There were no obvious differences in the number of primary cilia in single Oc-Cre; Kif3a^{flox/+} and Kif3a^{flox/null} heterozygous osteoblasts; however, a marked reduction of cilia formation was observed in conditional Kif3a^{Oc-cKO}-null osteoblasts compared to control cells.

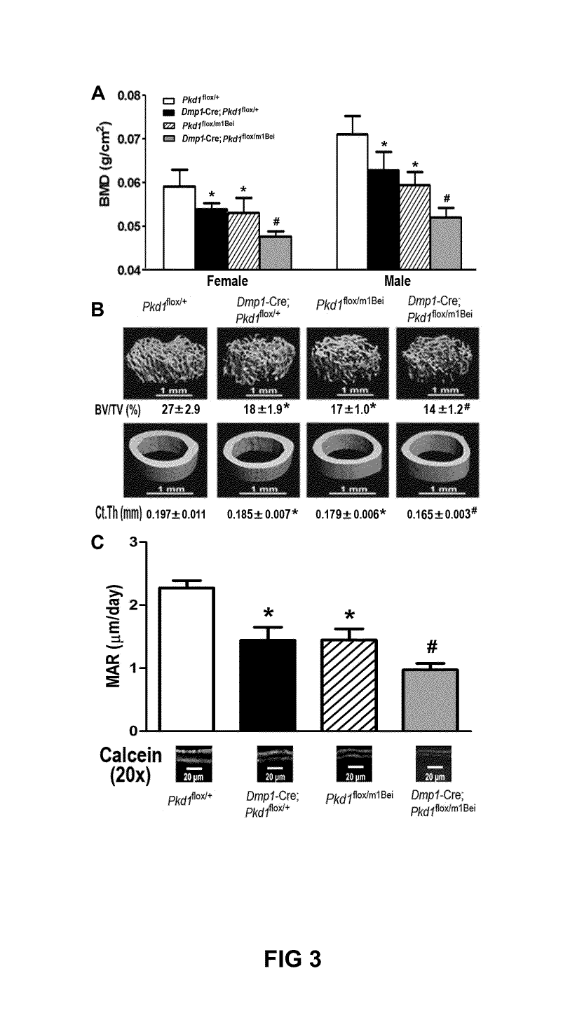

No change in body weight, lean body mass, or fat mass in Pkd1^{Dmp1-cKO} compared to controls (data not shown) was observed. In contrast, we observed a reduction of BMD of 9˜12% in both female and male Pkd1^{m1Bei/flox} and Dmp1-Cre; Pkd1^{flox/+} heterozygous mice at 6 weeks of age and a greater reduction in BMD of 19˜27% in Pkd1^{Dmp1-cKO} mice compared with age-matched control mice (Pkd1^{flox/+}) (FIG. 3A). μCT analysis revealed that the lower bone mass in single Pkd1^{m1Bei/flox} and Dmp1-Cre; Pkd1^{flox/+} heterozygous mice was caused by a reduction in trabecular bone volume (37% and 32%, respectively) and cortical bone thickness (10% and 6%, respectively) (FIG. 3B). Pkd1^{Dmp1-cKO} had greater loss in both trabecular (48%) and cortical bone (17%) (FIG. 3B), indicating that the conditional deletion of Pkd1 on the Pkd1^{m1Bei} background resulted in additional bone loss. These reductions in bone volume were associated with a significant decrease in mineral apposition rate in single Pkd1^{m1Bei/flox} and Dmp1-Cre; Pkd1^{flox/+} heterozygous mice compared with age-matched control mice and an even greater reduction in Pkd1^{Dmp1-cKO} mice (FIG. 3C).

Referring to FIG. 3. Dmp1-Cre-mediated somatic loss of Pkd1 leads to osteopenia. (A) Effects of Dmp1-Cre-mediated Pkd1^{Δflox} allele on bone mineral density (BMD) at 6 weeks of age. Similar to Beier Pkd1 heterozygous mice (Pkd1^{m1Bei/flox}), there was approximately 9˜12% reduction of BMD in both female and male single-excised floxed Pkd1 heterozygous mice (Dmp1-Cre; Pkd1^{flox}) compared with age-matched control mice (Pkd1^{flox/+}), and an even greater reduction (19˜27%) in double heterozygous Dmp1-Cre; Pkd1^{flox/m1Bei} (Pkd1^{Dmp1-cKO}) mice, indicating an additive effect of global mutant and conditional deleted Pkd1 alleles on loss of bone mass. (B) Effects of Dmp1-Cre-mediated Pkd1^{Δflox} allele on bone structure of femurs. μCT analysis of the distal femoral metaphyses and midshaft diaphyses revealed that double heterozygous Pkd1^{Dmp1-cKO} mice had greater loss in both trabecular and cortical bone than did single Dmp1-Cre; Pkd1^{flox/+} and Pkd1^{m1Bei/flox} heterozygous mice, consistent with additive effects of global mutant and conditional deleted Pkd1 alleles on bone structure and a direct role of Pkd1 in bone in osteocytes. (C) Effects of Dmp1-Cre-mediated Pkd1^{Δflox} allele on bone mineral apposition rate. There was a significant decrease in mineral apposition rate in single Pkd1^{m1Bei/flox} and Dmp1-Cre; Pkd1^{flox/+} heterozygous mice compared with age-matched control mice and an even greater reduction in double heterozygous Dmp1-Cre; Pkd1^{flox/m1Bei} mice, indicating an additive effect of global mutant and conditional deleted Pkd1 alleles to impair osteoblast-mediated bone formation. Data represent the mean±SD from five to six individual mice. *Indicates significant difference from control (Pkd1^{flox/+}), and ^{#} indicates significant difference from single heterozygous Dmp1-Cre; Pkd1^{flox/+} and Pkd1^{flox/m1Bei} mice at P<0.05, respectively.

To investigate whether combined Pkd1^{Δflox} and Pkd1^{m1Bei} deficiency resulted in additive effects on gene expression profiles in bone, we examined by real-time RT-PCR the expression levels of a panel of osteoblast lineage-, osteoclast-, and chondrocyte-related mRNAs from the tibias of 6-week-old control, heterozygous Pkd1 deficient (Dmp1-Cre; Pkd1^{flox/+} and Pkd1^{flox/m1Bei}), and Dmp1-Cre; Pkd1^{flox/m1Bei} mice (Table 1). Bone derived from heterozygous Dmp1-Cre; Pkd1^{flox/+} and Pkd1^{flox/m1Bei} mice had measurable reductions in the osteoblast-lineage gene transcripts, including Runx2-II, total Runx2, Osteocalcin, Osteopontin, Bsp, Osteoprotegerin (Opg), Mmp13, Dmp1, and Phex mRNA levels, compared to control mice. Significantly greater reductions of Runx2-II, total Runx2, Osteocalcin, Osteopontin, Bsp, RankL, Mmp13, Dmp1, and Phex were observed in Pkd1^{Dmp1-cKO} null mice. In this regard, the Opg/RankL expression ratio was increased in a gene dose-dependent manner (Table 1). Consistent with a ratio of Opg/RankL that favored the reduced osteoclastogenesis, bone expression of Trap and Mmp9, markers of bone resorption, were also reduced in heterozygous Pkd1-deficient mice and to a greater extent in Pkd1^{Dmp1-cKO} null mice (Table 1). In contrast, the transcription of Sost/sclerostin, an osteocyte-derived negative regulator of bone formation, was significantly increased in heterozygous Pkd1-deficient and Pkd1^{Dmp1-cKO} null mice compared to control mice, and transcripts of chondrocyte-related genes did not differ between heterozygous Pkd1-deficient and Pkd1^{Dmp1-cKO} null mice (Table 1). In addition, PPARγ, an adipocyte transcription factor, and adipocyte markers such as adipocyte fatty acid-binding protein 2 (aP2) were also increased in tibias of Pkd1-deficient mice in a Pkd1 gene dosage-related manner (Table 1).

TABLE 1
Gene-expression profiles in 6-week-old mice

| | | Dmp1-Cre; | | Dmp1-Cre; | |
|---|---|---|---|---|---|
| Gene | Accession no. | Pkd1^{flox/+} | Pkd1^{flox/m1Bei} | Pkd1^{flox/m1Bei} | P-value |
| Osteoblast lineage | | | | | |
| Runx2-II | NM_009820 | 0.77 ± 0.07* | 0.75 ± 0.06* | 0.52 ± 0.17*^{, #} | 0.0007 |
| Runx2-I | D14636 | 1.06 ± 0.63 | 0.96 ± 0.31 | 0.94 ± 0.17 | 0.9504 |
| Runx2 | NM_009820 | 0.73 ± 0.08* | 0.69 ± 0.13* | 0.47 ± 0.15*^{, #} | <0.0001 |
| Osteocalcin | NM_007541 | 0.69 ± 0.12* | 0.66 ± 0.11* | 0.41 ± 0.11*^{, #} | 0.0002 |
| Osteopontin | AF515708 | 0.72 ± 0.14* | 0.69 ± 0.16* | 0.39 ± 0.19*^{, #} | 0.0001 |
| Bsp | NM_008318 | 0.70 ± 0.12* | 0.71 ± 0.07* | 0.56 ± 0.11*^{, #} | <0.0001 |
| Opg | MMU94331 | 0.75 ± 0.12* | 0.72 ± 0.13* | 0.65 ± 0.12* | 0.0200 |
| Rank ligand | NM_011613 | 0.70 ± 0.14* | 0.71 ± 0.13* | 0.41 ± 0.17*^{, #} | 0.0003 |
| Mmp13 | NM_008607 | 0.66 ± 0.14* | 0.61 ± 0.12* | 0.37 ± 0.16*^{, #} | <0.0001 |
| Dmp1 | MMU242625 | 0.63 ± 0.17* | 0.60 ± 0.16* | 0.29 ± 0.05*^{, #} | 0.0004 |
| Phex | NM_011077 | 0.67 ± 0.14* | 0.68 ± 0.05* | 0.54 ± 0.11*^{, #} | <0.0001 |
| Sost | NM_024449 | 1.62 ± 0.47* | 1.67 ± 0.11* | 1.64 ± 0.31* | 0.0013 |
| Osteoclast | | | | | |
| Trap | NM_007388 | 0.65 ± 0.16* | 0.63 ± 0.21* | 0.31 ± 0.12*^{, #} | 0.0004 |
| Mmp9 | NM_013599 | 0.71 ± 0.11* | 0.77 ± 0.09* | 0.33 ± 0.09* | <0.0001 |
| Chondrocyte | | | | | |
| Collagen II | NM_031163 | 1.28 ± 0.89 | 1.30 ± 0.85 | 0.97 ± 0.38 | 0.7666 |
| VegfA | NM_009505 | 1.02 ± 0.55 | 1.01 ± 0.29 | 1.19 ± 0.62 | 0.8889 |
| Adipocyte | | | | | |
| PPARγ | NM_009505 | 1.41 ± 0.35* | 1.56 ± 0.38* | 1.76 ± 0.41* | 0.00168 |
| aP2 | NM_024406 | 1.46 ± 0.16* | 1.57 ± 0.23* | 1.95 ± 0.26*^{, #} | 0.0058 |

Data are mean ± SD from five to six tibias of 6-week-old individual mice and expressed as the x-fold changes relative to the housekeeping gene cyclophilin A subsequently normalized to control (Pkd1^{flox/+}) mice.
*Indicates significant difference from control (Pkd1^{flox/+}), and
^{#}indicates significant difference from single heterozygous Dmp1-Cre; Pkd1^{flox/+} and Pkd1^{flox/m1Bei} mice at P < 0.05, respectively.

Changes in gene expression in bone correlated with alterations in serum biomarkers. In this regard, Control Pkd1^{flox/+} mice had reduced osteoblastic and osteoclastic markers as a function of age, consistent with an age-dependent decrease in bone formation and resorption (Table 2). At 6 weeks of age, Pkd1^{Dmp1-cKO} null mice had a further reduction in both osteoblast and osteoclast markers, evidenced by decreased Osteocalcin (106±12 vs. 39±7 ng/ml), RANK-L (179±29 vs. 115±28 pg/ml), and TRAP (3.4±0.73 vs. 2.0±0.29 U/1) compared to age-matched control Pkd1^{flox/+} mice. At 16 weeks of age, a reduction in Osteocalcin was not observed in Pkd1^{Dmp1-cKO} null mice, but decreases in RANK-L (110±14 vs. 91±7 pg/ml), and TRAP (2.7±0.34 vs. 2.1±0.42 U/l) remains the same compared to age-matched control Pkd1^{flox/+} mice. In addition, the RANK-L/OPG ratio, which is an indicator of osteoclastogenesis, was reduced by approximately 43% at 6 weeks of age and 6% at 16 weeks of age in Pkd1^{Dmp1-cKO} null mice compared to age-matched control Pkd1^{flox/+} mice, consistent with the reduction in osteoclastic markers (Table 2). These data suggest that the conditional loss of Pkd1 in osteocytes results in diminished osteoblast and osteoclast function and consequent low-turnover osteopenia. In contrast, Pkd1^{Dmp1-cKO} null mice had no effect on serum calcium and phosphorus levels at either 6 or 16 weeks (Table 2).

TABLE 2
Biochemistry analysis of serum in 6- and 16-week-old mice.

| Serum values | Pkd1^{flox/+} | Pkd1^{Dmp1-cKO} |
|---|---|---|
| BUN(mg/dl) | 31 ± 4.4 | 30 ± 4.8 |
| | 29 ± 5.1 | 28 ± 3.5 |
| Ca (mg/dl) | 8.9 ± 0.26 | 9.1 ± 0.27 |
| | 8.7 ± 0.39 | 9.0 ± 0.22 |
| P (mg/dl) | | |
| 6 weeks | 8.1 ± 0.33 | 8.0 ± 0.48 |
| 16 weeks | 7.6 ± 0.61 | 7.2 ± 0.34 |
| Osteocalcin (ηg/ml) | | |
| 6 weeks | 106 ± 12 | 39 ± 7* |
| 16 weeks | 14.4 ± 5.1 | 12.3 ± 5.5 |
| OPG (ηg/ml) | | |
| 6 weeks | 2.4 ± 0.33 | 2.7 ± 0.53 |
| 16 weeks | 2.5 ± 0.34 | 2.2 ± 0.42 |
| RankL (pg/ml) | | |
| 6 weeks | 179 ± 29 | 115 ± 28* |
| 16 weeks | 110 ± 14 | 91 ± 7* |
| TRAP (U/L) | | |
| 6 weeks | 3.4 ± 0.73 | 2.0 ± 0.29* |
| 16 weeks | 2.7 ± 0.34 | 2.1 ± 0.42* |

Data are mean ± S. from five to six individual mice.
*Indicates significant difference from control mice at P < 0.05, respectively.
Osteocalcin is produced by osteoblasts, and TRAP is produced by osteoclasts.

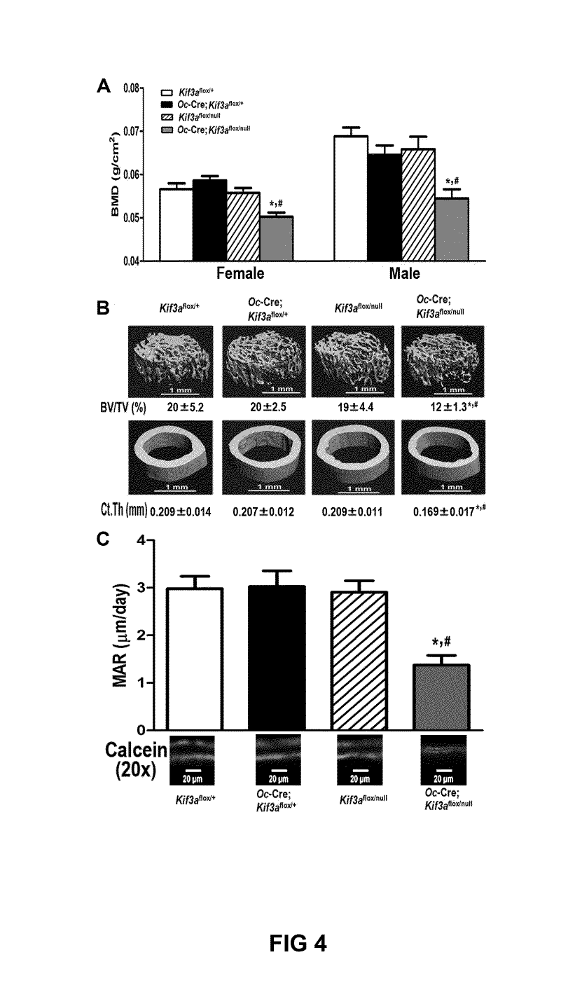

No differences was observed in body weight, lean body mass, or fat mass between any of the three Kif3a-deficient mice compared to controls at 6 weeks-of-age (data not shown). It was found that single heterozygous Oc-Cre; Kif3a^{flox/+} or Kif3a^{flox/null} mice had no demonstrable bone abnormalities (FIG. 4). Bone mineral density (BMD) and bone structure (FIGS. 4A and 4B), as well as mineral apposition rates (MAR) (FIG. 4C) were not different between single heterozygous Oc-Cre; Kif3a^{flox/+} or Kif3a^{flox/null} and age-matched control mice (Kif3a^{flox/+}). In addition, bone samples from Oc-Cre; Kif3a^{flox/+} or Kif3a^{flox/null} mice had no detectable changes in markers of either osteoblast or osteoclast gene expression (Table 3). However, Kif3a^{flox/null} mice had significant reductions in adipocyte-related markers, including peroxisome proliferator-activated receptor gamma (PPARγ), adipocyte-specific fatty acid binding protein (aP2), and lipoprotein lipase (Lpl) in long-bone samples (Table 3).

TABLE 3
Gene-expression profiles in 6-week-old mice.

| Gene | Accession no. | Oc-Cre; Kif3a^{flox/+} | Kif3a^{flox/null} | Oc-Cre; Kif3a^{flox/null} | p-value |
|---|---|---|---|---|---|
| Osteoblast lineage | | | | | |
| Runx2 | NM_009820 | 1.10 ± 0.39 | 0.96 ± 0.16 | 0.60 ± 0.13*^{,#} | 0.0124 |
| Osterix | NM_130458 | 0.99 ± 0.24 | 0.96 ± 0.24 | 0.62 ± 0.11*^{,#} | 0.0058 |
| Osteocalcin | NM_007541 | 1.13 ± 0.19 | 1.12 ± 0.27 | 0.71 ± 0.15*^{,#} | 0.0102 |
| Opg | MMU94331 | 1.18 ± 0.16 | 1.06 ± 0.16 | 0.94 ± 0.17 | 0.1310 |
| Rank ligand | NM_011613 | 0.97 ± 0.22 | 0.96 ± 0.16 | 0.66 ± 0.09*^{,#} | 0.0236 |
| Dmp1 | MMU242625 | 1.11 ± 0.12 | 1.12 ± 0.34 | 0.65 ± 0.08*^{,#} | 0.0054 |
| Osteoclast | | | | | |
| Trap | NM_007388 | 1.19 ± 0.18 | 1.17 ± 0.12 | 0.73 ± 0.09*^{,#} | 0.0002 |
| Mmp9 | NM_013599 | 1.11 ± 0.11 | 1.04 ± 0.12 | 0.84 ± 0.11*^{,#} | 0.0032 |
| Adipocyte | | | | | |
| PPARγ | NM_009505 | 0.81 ± 0.11 | 0.62 ± 0.12* | 1.09 ± 0.23^{#} | 0.0031 |
| aP2 | NM_024406 | 0.86 ± 0.11 | 0.71 ± 0.10* | 1.08 ± 0.22^{#} | 0.0112 |
| Lpl | NM_008509 | 0.75 ± 0.13 | 0.51 ± 0.12* | 1.51 ± 0.54*^{,#} | 0.0001 |

Data are mean ± S.D. from 5-6 tibias of 6-week-old individual mice and expressed as the fold changes relative to the housekeeping gene cyclophilin A subsequently normalized to wild-type mice.
*indicates significant difference from control Kif3a^{flox/+} mice, and
^{#}indicates significant difference from single heterozygous Oc-Cre;Kif3a^{flox/+}Kif3a^{+/Δ} and Kif3a^{flox/null} mice at p < 0.05, respectively.

In contrast, we observed a significant reduction in BMD of 11˜21% in both female and male conditional Kif3a^{Oc-cKO}-null mice at 6 weeks-of-age compared with age-matched control mice (Kif3a^{flox/+}) (FIG. 4A). μCT analysis revealed that the lower bone mass in male conditional Kif3a^{Oc-cKO}-null mice was caused by reduced trabecular bone volume (BV/TV, 42%) and cortical bone thickness (Ct.Th, 17%) (FIG. 4B). These reductions in bone volume and cortical thickness were associated with a 2-fold decrease in periosteal mineral apposition rate (MAR) in male conditional Kif3a^{Oc-cKO}-null mice compared with age-matched control mice (FIG. 4C). To investigate the effects of the combined Kif3a^{Δflox} and Kif3a^{null} deficiency on gene expression profiles in bone, by real-time RT-PCR was conducted to determine the expression levels of a panel of osteoblast lineage-, osteoclast-, and adipocyte-related mRNAs from the tibias of 6-week-old male control and conditional Kif3a^{Oc-cKO}-null mice (Table 3). Bone derived from conditional Kif3a^{Oc-cKO}-null mice had significant reductions in osteoblast-lineage gene transcripts, including Runx2, Osterix, Osteocalcin, Rank ligand, and Dmp1 mRNA levels, but no obvious change was observed in Osteoprotegerin (Opg) expression compared to control mice. In this regard, the Opg/RankL expression ratio was increased in conditional Kif3a^{Oc-cKO}-null mice (Table 3). Consistent with a ratio of Opg/RankL that favored the reduced osteoclastogenesis, bone expression of Trap and Mmp9, markers of bone resorption, were also reduced in conditional Kif3a^{Oc-cKO}-null mice (Table 3). In contrast, PPARγ, an adipocyte transcription factor, and adipocyte markers such as aP2 and Lpl were significantly increased in tibias of conditional Kif3a^{Oc-cKO}-null mice compared to single heterozygous Kif3a^{flox/null} mice, and a greater increase of Lpl was observed in conditional Kif3a^{Oc-cKO}-null mice than that in control Kif3a^{flox/+} mice (Table 3).

Now referring to FIG. 4. Oc-Cre-mediated somatic deletion under Kif3a-deficient background leads to osteopenia. Effects of global and/or Oc-Cre-mediated deletion of Kif3a on (A) bone mineral density (BMD), (B) bone structure of femurs, and (C) bone mineral apposition rate (MAR) at 6 weeks-of-age. There was no bone loss in either female or male single heterozygous Oc-Cre; Kif3a^{flox/+} and Kif3a^{flox/null} mice, evidenced by normal BMD, BV/TV, CtTh, or MAR compared with age-matched control mice (Kif3a^{flox/+}). In contrast, there was a significant reduction in BMD in both female and male conditional Kif3a^{Oc-cKO}-null mice compared with age-matched control mice (Kif3a^{flox/+}). μCT analysis revealed that the lower bone mass in male conditional Kif3a^{Oc-cKO}-null mice was the result of a reduction in trabecular BV/TV and cortical CtTh. These reductions in bone mass and structure were associated with a 2-fold decrease in MAR in male conditional Kif3a^{Oc-cKO}-null mice compared with age-matched control mice. Data represent the mean±S.D. from five to six individual mice. *significant difference from control (Kif3a^{flox/+}) and ^{#} significant difference from single heterozygous Oc-Cre; Kif3a^{flox/+} and Kif3a^{flox/null} mice, P<0.05, respectively.

Changes in gene expression in bone correlated with alterations in serum biomarkers. In this regard, control Kif3a^{flox/+} mice had reduced osteoblastic and osteoclastic markers as a function of age, consistent with an age-dependent decrease in bone formation and resorption. At 6 weeks-of-age, Kif3a^{Oc-cKO}-null mice exhibited levels of the osteoblast markers, Osteocalcin (59±10 vs. 42±8 ηg/ml) and Rank ligand (RankL) (122±24 vs. 79±28 pg/ml), as well as the osteoclastic marker tartrate-resistant acid phosphatase (TRAP) (5.6±0.29 vs. 4.5±0.43 U/L) compared to age-matched control Kif3a^{flox/+} mice. At 24 weeks-of-age, a reduction in Osteocalcin was no longer observed in Kif3a^{Oc-cKO}-null mice, but RankL (95±22 vs. 54±16 pg/ml) and TRAP (1.7±0.32 vs. 0.8±0.11 U/L) remained reduced compared to age-matched control Kif3a^{flox/+} mice. While there was no difference in the osteoprotegerin (OPG) level, the RankL/OPG ratio, which is an indicator of osteoclastogenesis, was reduced by approximately 29% at 6 weeks-of-age and 47% at 24 weeks-of-age in Kif3a^{Oc-cKO-} null mice compared to age-matched control Kif3a^{flox/+} mice. These data suggest that conditional deletion of Kif3a in osteoblasts results in diminished osteoblast-mediated bone formation and osteoclast-mediated bone resorption, resulting in low-turnover osteopenia. Kif3a^{Oc-cKO}-null mice had no change in serum urea nitrogen (BUN), calcium, or phosphorus levels at either 6 or 24 weeks (data not shown).

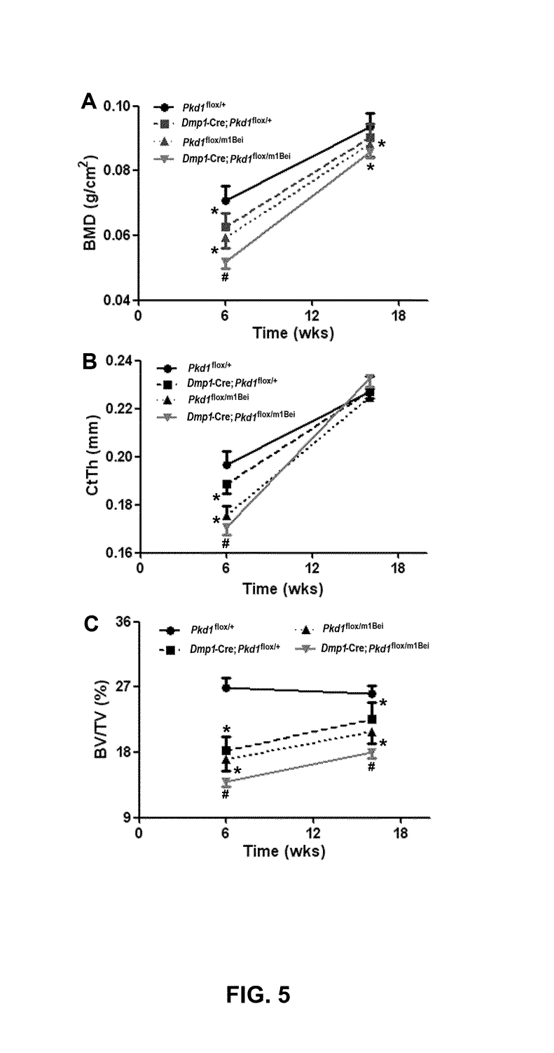

Compared with control mice, there was an age-dependent partial recovery of Bone Mineral Density (BMD) from 27%, 16%, and 12% reduction at 6 weeks of age to 10%, 6%, and 4% reduction at 16 weeks of age in Pkd1^{Dmp1-cKO}, Pkd^{1flox/m1Bei}, and Dmp1-Cre; Pkd1^{flox/+} mice, respectively, indicating age-dependent effects that attenuate the effects of mutant Pkd1 on bone mass (FIG. 5A). CT analysis revealed that the increase in bone mass was caused by a recovery in cortical bone thickness. Indeed, the differences in cortical bone thickness observed at 6 weeks of age were no longer significant in the four genotypes at 16 weeks of age (FIG. 5B). In contrast, there was less recovery of bone volume/trabecular volume (BV/TV) with age. In this regard, BV/TV remained significantly lower in single Pkd1^{flox/m1Bei} and Dmp1-Cre; Pkd1^{flox/+} heterozygous mice, as well as in Pkd1^{Dmp1-cKO} mice compared to control mice at 16 weeks of age (FIG. 5C), suggesting a site-specific interaction between Pkd1 mutations and age-dependent changes in bone structure. Moreover, we found that the mechanism of cortical bone recovery resulted from alterations in bone geometry and led to a compensatory increase in bone mechanical properties in 16-week-old of Pkd1^{Dmp1-cKO} mice. In this regard, single Pkd1^{flox/m1Bei} heterozygous and conditional Pkd1^{Dmp1-cKO} null mice showed a gene dose dependent decrease in total bone area but no difference in cortical bone area when compared with age-matched control Pkd1^{flox/+} mice (Table 4), consistent with a reduction in the size of the marrow cavity resulting from a smaller midshaft diameter compared with control mice. However, there was a significant reduction in moment of inertia, Ix and c (a distance from the neutral axis to the plane where the load is applied), in Pkd1^{Dmp1-cKO} null mice, but no differences in single Pkd1^{flox/m1Bei} and Dmp1-Cre; Pkd1^{flox/+} heterozygous mice compared to control mice (Table 4). To examine whether changes of femoral bone geometry may affect bone mechanical properties, a 3-point bending experiments was conducted (Table 5). Compared with the control mice, conditional Pkd1^{Dmp1-cKO} null mice had a higher maximum stress only in 3-point bending but no significant differences in bending stiffness, maximum force, or energy to failure (Table 5), indicating that the changes of bone geometry and bone structure at 16 weeks of age may preserve bone strength in conditional Pkd1^{Dmp1-cKO} null mice. There was no difference in these parameters between single heterozygous mice and control mice (Table 5).

TABLE 4
Femur bone geometry in 18-week-old mice

| | | Dmp1-Cre; | | Dmp1-Cre; |
|---|---|---|---|---|
| Parameter | Pkd1^{flox/+} | Pkd1^{flox/+} | Pkd1^{flox/m1Bei} | Pkd1^{flox/m1Bei} |
| Total area (mm²) | 2.1 ± 0.16 | 2.2 ± 0.14 | 1.9 ± 0.11* | 1.7 ± 0.13*^{,#} |
| Cortical area (mm²) | 1.03 ± 0.09 | 1.08 ± 0.08 | 0.99 ± 0.05 | 0.98 ± 0.08 |
| Moment of inertia, Ix (mm⁴) | 0.19 ± 0.03 | 0.21 ± 0.02 | 0.17 ± 0.02 | 0.14 ± 0.02*^{,#} |
| c (mm) | 0.71 ± 0.05 | 0.70 ± 0.04 | 0.68 ± 0.04 | 0.63 |
| | | | | 0.03*^{,#} |

c, a distance from the neutral axis to the plane where the load is applied.
Data represent the mean ± SD from five to six individual mice.
*indicates significant difference from control (Pkd1^{flox/+}) and Dmp1-Cre; Pkd1^{flox/+} mice and
^{#}indicates significant difference from Pkd1^{flox/m1Bei} mice at P < 0.05, respectively.

TABLE 5
Femur biomechanical properties in 18-week-old mice

| | | Dmp1-Cre; | | Dmp1-Cre; |
|---|---|---|---|---|
| Parameter | Pkd1^{flox/+} | Pkd1^{flox/+} | Pkd1^{flox/m1Bei} | Pkd1^{flox/m1Bei} |
| Stiffness (N/mm) | 210 ± 35 | 174 ± 34 | 203 ± 41 | 179 ± 46 |
| Maximum force | 22 ± 3.8 | 23 ± 3.9 | 23 ± 2.9 | 22 ± 3.0 |
| (N) | | | | |
| Maximum stress | 108 ± 16 | 109 ± 19 | 122 ± 17 | 132 ± 14* |
| (Mpa) | | | | |
| Energy to failure | 9.7 ± 2.1 | 9.2 ± 1.9 | 8.4 ± 1.6 | 8.1 ± 2.1 |
| (N-mm) | | | | |

Data represent the mean ± SD from five to six individual mice.
*indicates significant difference from control (Pkd1^{flox/+}) and Dmp1-Cre; Pkd1^{flox/+} mice at P < 0.05, respectively.

Referring to FIG. 5. Age-dependent effects of Dmp1-Cre-mediated Pkd1^{Δflox} allele on bone mass. (A) Age-dependent effects of Dmp1-Cre-mediated Pkd1^{Δflox} allele on femoral Bone Mineral Density. (B) Age-dependent effects of Dmp1-Cre-mediated Pkd1^{Δflox} allele on trabecular bone volume of distal femoral metaphyses. (C) Age-dependent effects of Dmp1-Cre-mediated Pkd1^{Δflox} allele on cortical thickness of femoral midshaft diaphyses. Compared with control mice, there was an age-dependent partial recovery of BMD in Dmp1-Cre; Pkd1^{flox/+}, Pkd1^{flox/m1Bei}, and Pkd1^{Dmp1-cKO} mice, respectively. μCT analysis revealed that the increase in bone mass was caused by a recovery in cortical bone thickness, but trabecular bone volume (BV/TV) remained significantly lower in these Pkd1-deficient mice at 16 weeks-of-age. Data represent the mean±S.D. from 5-6 individual mice. * indicates significant difference from control (Pkd1^{flox/+}), and ^{#}indicates significant difference from single heterozygous Dmp1-Cre; Pkd1^{flox/+} and Pkd1^{flox/m1Bei} mice at p<0.05, respectively.

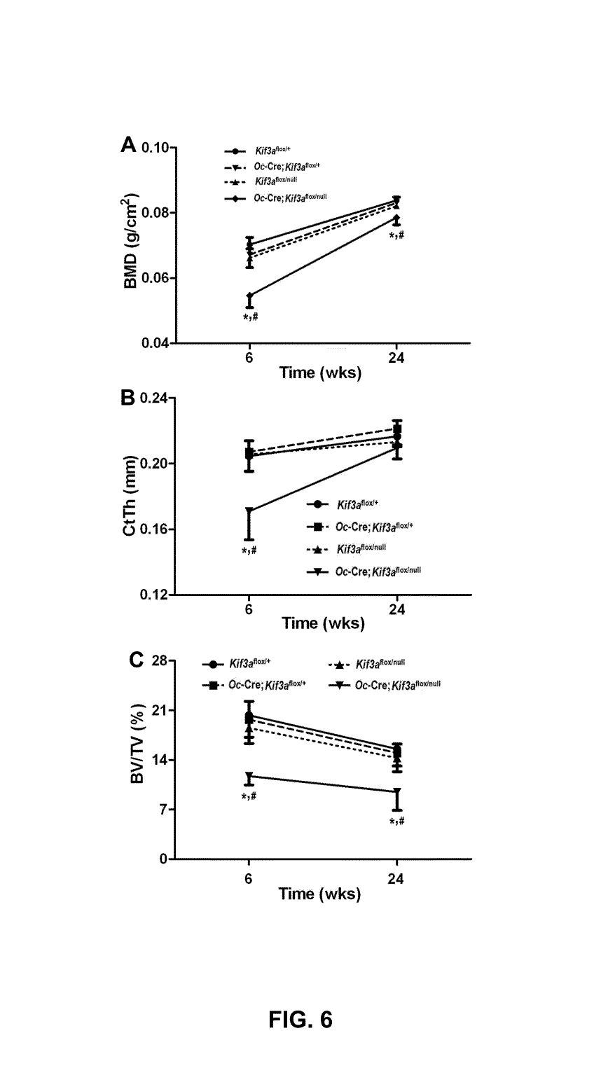

No differences was observed in bone mass and bone structure between single heterozygous Oc-Cre; Kif3a^{flox/+} or Kif3a^{flox/null} mice and control Kif3a^{flox/+} mice from 6 to 24 weeks-of-age. However, an age-dependent partial recovery of Bone Mineral Density was observed from 21% reduction at 6 weeks-of-age to 7% reduction at 24 weeks-of-age in male conditional Kif3a^{Oc-cKO}-null mice compared to age-matched control mice, indicating age-dependent effects that attenuate the effects of deleted Kif3a on bone mass (FIG. 6A). μCT analysis revealed that the increase in bone mass was caused by a recovery in cortical bone thickness. Indeed, the differences in cortical bone thickness observed at 6 weeks-of-age were no longer significant in the four genotypes at 24 weeks-of-age (FIG. 6B). In contrast, there was less recovery of BV/TV with age in Kif3a^{Oc-cKO} mice. In this regard, BV/TV remained significantly lower in Kif3a^{Oc-cKO} mice compared to control mice at 24 weeks-of-age (FIG. 6C), a finding that suggests a site-specific interaction between Kif3a deficiency and age-dependent changes in bone structure. Moreover, it was found that the mechanism of Bone Mineral Density and cortical bone thickness recovery resulted from alterations in bone geometry and led to a compensatory increase in bone mechanical properties between 6- and 24-week-old of Kif3a^{Oc-cKO} mice (Table 6). Also, conditional Kif3a^{Oc-cKO}-null mice showed an increase in total bone area, moment of inertia (Ix), and c (a distance from the neutral axis to the plane where the load is applied) at 6 weeks-of-age. Kif3a^{Oc-cKO}-null mice exhibited no difference in cortical bone area compared with age-matched control Kif3a^{flox/+} mice (Table 6), but had an increment in the size of the marrow cavity resulting from a greater midshaft diameter compared with control mice. However, these structural differences were no longer evident in 24-week-old Kif3a^{Oc-cKO} mice, because of a recovery in femoral midshaft diameter and the size of the marrow cavity with age (Table 6).

TABLE 6
Femur bone geometry and biomechanical properties in 6- and 24-week-old
mice

| Parameter | Control | Kif3a^{Oc-ckO} |
|---|---|---|
| Total area (mm²) | | |
| 6 weeks | 1.9 ± 0.11 | 2.1 ± 0.09* |
| 24 weeks | 2.0 ± 0.14 | 2.0 ± 0.07 |
| Cortical area (mm²) | | |
| 6 weeks | 0.58 ± 0.16 | 0.62 ± 0.08 |
| 24 weeks | 0.59 ± 0.05 | 0.57 ± 0.12 |
| Moment of inertia (mm⁴) | | |
| 6 weeks | 0.15 ± 0.03 | 0.18 ± 0.02* |
| 24 weeks | 0.15 ± 0.02 | 0.15 ± 0.03 |
| c (mm) | | |
| 6 weeks | 0.66 ± 0.02 | 0.70 ± 0.02* |
| 24 weeks | 0.64 ± 0.03 | 0.65 ± 0.03 |
| Stiffness (N/mm) | | |
| 6 weeks | 84 ± 22 | 104 ± 22 |
| 24 weeks | 159 ± 25 | 122 ± 22 |
| Maximum force (N) | | |
| 6 weeks | 15 ± 2.5 | 17 ± 2.1 |
| 24 weeks | 17 ± 1.9 | 16 ± 0.6 |
| Elastic Modulus (GPa) | | |
| 6 weeks | 4.1 ± 1.8 | 2.7 ± 0.9* |
| 24 weeks | 4.5 ± 2.9 | 5.4 ± 2.7 |

c (mm), a distance from the neutral axis to the plane where the load is applied.
Data represent the mean ± S.D. from four individual mice.
*significant difference from 6-week-old control mice, P < 0.05.

Referring to FIG. 6. Age-dependent effects of global and/or Oc-Cre-mediated conditional deletion of Kif3a on bone mass and structure. Age-dependent effects of global and Oc-Cre-mediated somatic deletion of Kif3a on (A) femoral BMD, (B) trabecular bone volume of distal femoral metaphyses, (C) cortical bone thickness of femoral midshaft diaphyses. Compared with control (Kif3a^{flox/+}) mice, there was an age-dependent partial recovery of Bone Mineral Density in male conditional Kif3a^{Oc-cKO}-null mice from 6 to 24 weeks-of-age. CT analysis revealed that the increase in bone mass resulted from a complete recovery in cortical bone thickness, but BV/TV remained significantly lower in male conditional Kif3a^{Oc-cKO}-null mice at 24 weeks-of-age. However, single heterozygous Oc-Cre; Kif3a^{flox/+} and Kif3a^{flox/null} mice had normal bone mass and bone structure compared with age-matched controls. Data represent the mean±S.D. from five to six individual mice. *significant difference from control (Kif3a^{flox/+} and ^{#} significant difference from single heterozygous Oc-Cre; Kif3a^{flox/+} and Kif3a^{flox/null} mice, P<0.05, respectively.

To examine whether changes of femoral bone geometry may affect bone mechanical properties, we used these femurs to perform three-point bending experiments (Table 6). At 6 weeks-of-age, conditional Kif3a^{Oc-cKO}-null mice had a lower elastic modulus in three-point bending experiments but no significant differences in bending stiffness or maximum force compared with age-matched control mice (Table 6), indicating that the changes in bone geometry and bone structure at 6 weeks-of-age had an impact on bone strength in conditional Kif3a^{Oc-cKO}-null mice. Again, the recovery of bone geometry and structure in 24-week old Kif3a^{Oc-cKO} normalized these mechanical properties (Table 6).

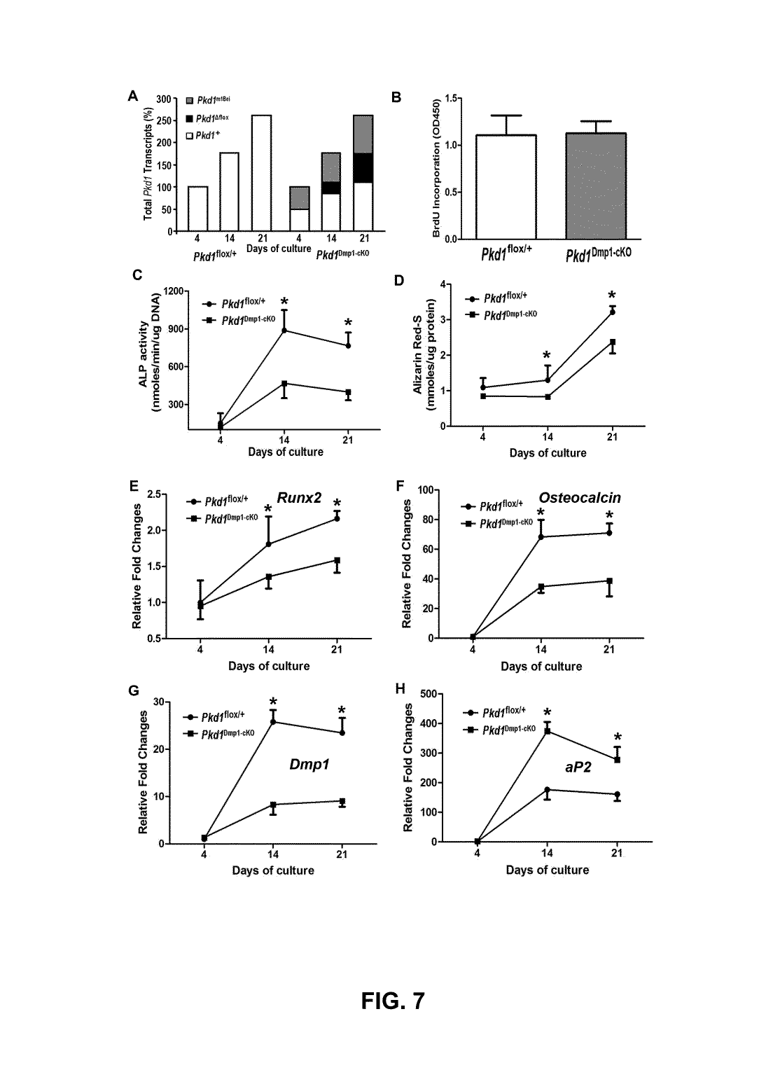

To determine the impact of conditional deleted Pkd1 on osteoblast function ex vivo cell proliferation and osteoblastic differentiation and gene expression profiles in primary osteoblast cultures derived from control and Pkd1^{Dmp1-cKO} null mice were examined. Control osteoblasts exhibited a time-dependent increase of total Pkd1 transcripts (wild type Pkd¹⁺ allele) during 21 days of osteogenic culture. In contrast, conditional Pkd^{1Dmp1-cKO} null osteoblasts showed a proportionate time-dependent increase of Pkd1 mutant alleles Pkd^{1Δflox} and Pkd^{1m1Bei} alleles consistent with 50%, 92%, and 150% inactivation of Pkd1 transcripts (FIG. 7A). Consistent with no Dmp1-Cre-mediated deletion of Pkd1 in the early stage of osteoblast culture (61), the Pkd1^{Dmp1-cKO} null osteoblasts showed no alterations on BrdU incorporation and cell proliferation (FIG. 7B). However, conditional Pkd^{1Dmp1-cKO} null osteoblasts displayed impaired osteoblastic differentiation and maturation, as evidenced by time-dependent lower alkaline phosphatase activity (FIG. 7C), diminished calcium deposition in extracellular matrix (FIG. 7D), and reduced osteoblastic differentiation markers, including Runx2, Osteocalcin, and Dmp1, compared to controls (FIG. 7E-7G). In agreement with increased adipogenic activity in vivo, the cultured primary calvarial cells under osteogenic condition exhibited a marked increase of adipocyte markers such as aP2 (FIG. 7H), suggesting impairment of osteogenesis and enhancement of adipogenesis in Pkd^{1Dmp1-K} null osteoblast cultures.

Referring to FIG. 7. Effects of Dmp1-Cre-mediated Pkd1 deletion on osteoblastic proliferation and maturation ex vivo. (A) Total Pkd1 transcripts by real-time RT-PCR from control and Pkd1^{Dmp1-cKO} osteoblasts during 21 days of culture. Control osteoblasts exhibited a time-dependent increase of total wild type Pkd¹⁺ allele, while conditional Pkd1^{Dmp1-cKO} null osteoblasts showed a proportionate time-dependent increase of Pkd1^{Δflox} and Pkd1 mutant alleles. (B) BrdU incorporation. There was no significant change in BrdU incorporation for 6 h between control and Pkd1^{Dmp1-cKO} osteoblasts, indicating Dmp1-Cre-mediated Pkd1 deletion did not affect proliferation of primary cultured osteoblasts. (C) ALP activity. Primary cultured Pkd1^{Dmp1-cKO} osteoblasts displayed time-dependent increments in alkaline phosphatase (ALP) activity during 21 days of culture, but the ALP activity was significantly lower at different time points compared with control Pkd1^{flox/+} osteoblasts. (D) Quantification of mineralization. Alizarin Red-S was extracted with 10% cetylpyridinium chloride and quantified as described in Experimental Procedures. Primary cultured Pkd1^{Dmp1-cKO} osteoblasts had time-dependent increments in Alizarin Red-S accumulation during 21 days of culture, but the accumulation was significantly lower at different time points compared with control Pkd1^{flox/+} osteoblasts. (E-H) Gene expression profiles by real-time RT-PCR. Primary cultured Pkd1^{Dmp1-cKO} osteoblasts in osteogenic differentiation media showed time-dependent increments in osteogenesis but significantly lower at different time points compared to control osteoblasts, evidenced by a significant reduction in osteoblastic markers including Runx2, Osteocalcin (Oc), and Dentin matrix protein 1 (Dmp1). In contrast, a marked increase of adipocyte markers such as aP2 at different time points was observed from the Pkd1^{Dmp1-cKO} osteoblasts under the same differentiation media when compared with control osteoblasts. Data are mean±SD from triple three independent experiments. * Indicates significant difference from control (Pkd1^{flox/+}) mice at P<0.05.

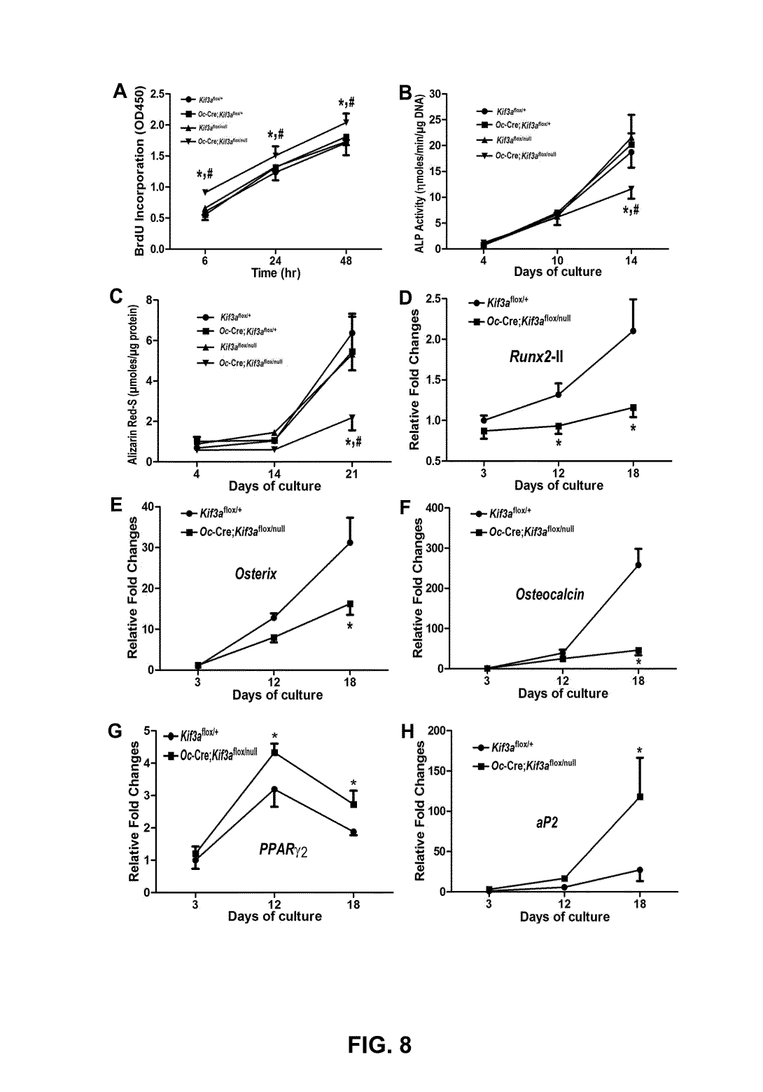

To determine the impact of conditional deletion of Kif3a on osteoblast function ex vivo, immortalized osteoblasts derived from E17.5 control Kif3a^{flox/+} mice, single heterozygous Oc-Cre; Kif3a^{flox/+} mice, single heterozygous Kif3a^{flox/null} mice, and conditional Kif3a^{Oc-cKO}-null mice were studied. Immortalized osteoblasts in culture undergo progressive alterations in cell proliferation and osteoblastic differentiation that recapitulates the osteoblastic developmental program. It was found that that heterozygous Kif3a deficient mice had no abnormalities of cell proliferation or osteoblastic differentiation. In this regard, the osteoblasts from Oc-Cre; Kif3a^{flox/+} mice and heterozygous Kif3a^{flox/null} mice exhibited a time-dependent increase of total BrdU incorporation during 48 h of osteogenic culture (FIG. 8A) as well as increased alkaline phosphatase (ALP) activity (a marker of differentiated osteoblasts) and calcium deposition in long-term cultures similar to controls (FIGS. 8B and 8C). It was found that conditional Kif3a^{Oc-cKO}-null osteoblasts had a higher BrdU incorporation than the other three groups, indicating a greater proliferation rate in Kif3a^{Oc-cKO}-null osteoblasts (FIG. 8A). In addition, Kif3a^{Oc-cKO}-null osteoblasts had impaired osteoblastic differentiation and maturation, as evidenced by culture duration-dependent reductions in ALP activity (FIG. 8B), diminished calcium deposition in extracellular matrix (FIG. 8C), and reduced osteoblastic differentiation markers, including Runx2-II, Osterix, and Osteocalcin, compared to control osteoblasts (FIG. 8D-8F). The immortalized primary calvarial osteoblasts derived from Kif3a^{Oc-cKO} mice also exhibited evidence for divergence from the osteoblastic development program, analogous to the increased adipogenic markers observed in vivo. Indeed, under osteogenic culture condition, Kif3a^{Oc-cKO} derived osteoblasts exhibited a marked increase of adipocyte markers such as PPARγ2 and aP2 (FIGS. 8G and 8H), suggesting that impairment of osteogenesis was associated with enhancement of adipogenesis in conditional Kif3a^{Oc-cKO}-null osteoblast cultures.

Now referring to FIG. 8. Effects of global and/or Oc-Cre-mediated conditional deletion of Kif3a on osteoblastic proliferation and maturation, as well as gene expression profiles ex vivo. (A) BrdU incorporation. There was a time-dependent increase of total BrdU incorporation during 48 h of osteogenic culture in osteoblasts from each of the four genotypes. However, a higher BrdU incorporation was observed in Kif3a^{Oc-cKO}-null osteoblasts compared to the other three groups of osteoblasts at the indicated time. (B) ALP activity. All osteoblasts from each of the four genotypes displayed time-dependent increments in ALP activity during 14 days of culture, but ALP activity was significantly lower in Kif3a^{Oc-cKO}-null osteoblasts compared to the other three groups of osteoblasts at day 14 of culture. (C) Quantification of mineralization. Alizarin Red-S was extracted with 10% cetylpyridinium chloride and quantified as described in Materials and Methods. All osteoblasts from each of the four genotypes had time-dependent increments in Alizarin Red-S accumulation during 21 days of culture, but the accumulation was significantly lower in Kif3a^{Oc-cKO}-null osteoblasts compared to the other three groups of osteoblasts at day 21 of culture. (D-H) Gene expression profiles by real-time RT-PCR. Immortalized Kif3a^{Oc-cKO}-null osteoblasts in osteogenic media showed time-dependent increments in osteogenesis during 18 days of culture but significantly lower at different time points compared to control (Kif3a^{flox/+}) osteoblasts, evidenced by a significant reduction in osteoblastic markers including Runx2, Osterix, and Osteocalcin (Oc). In contrast, a marked increase of adipocyte markers such as PPARγ2 and aP2 at different time points was observed from the Kif3a^{Oc-cKO}-null osteoblasts under the same osteogenic media when compared with control osteoblasts. Data are mean±S.D. from three independent experiments. *significant difference from control (Kif3a^{flox/+}) and ^{#} significant difference from single heterozygous Oc-Cre; Kif3a^{flox/+} and Kif3a^{flox/null} mice, P<0.05, respectively.

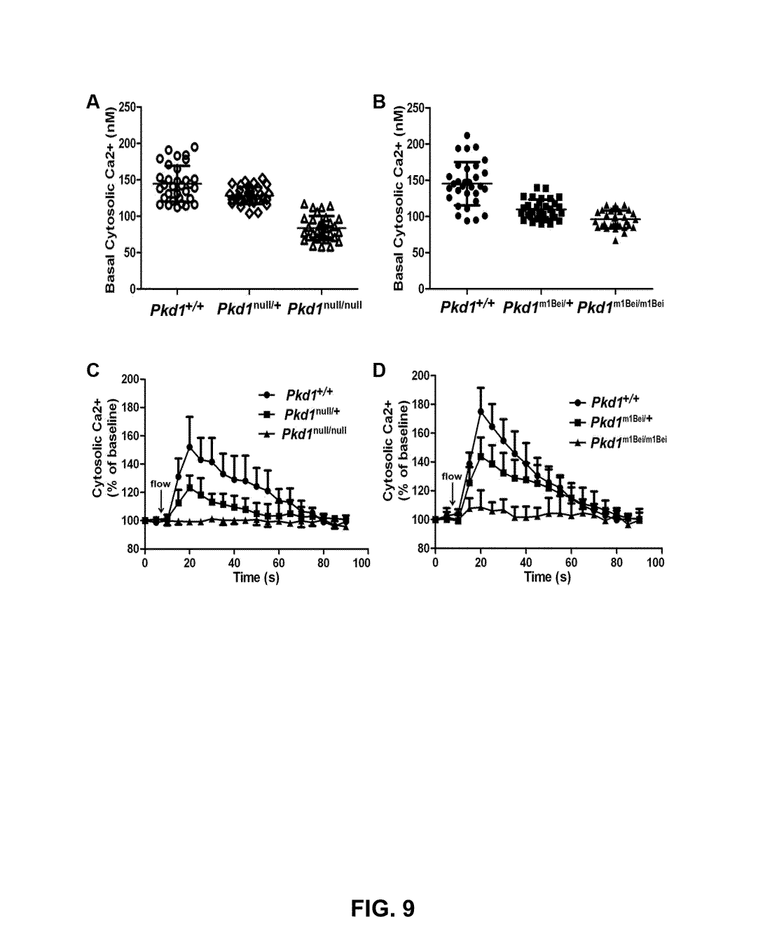

It was found that Pkd1 deletion/mutation had a gene dose effect on basal intracellular calcium ([Ca²⁺]ᵢ) concentration and flow-induced intracellular calcium response in immortalized Pkd1-deficient osteoblasts. In this regard, heterozygous Pkd1^{null/+} and Pkd^{1m1Bei/+} osteoblasts showed a significantly lower basal intracellular calcium ([Ca²⁺]ᵢ) concentration compared with wild type Pkd1^{+/+} cells, and homozygous Pkd1^{null/null} and Pkd^{1m1Bei/m1Bei} osteoblasts had greater reductions of basal [Ca²⁺]ᵢ compared with their respective heterozygous cells (FIGS. 9A and 9B). To study whether polycystin-1-mediated mechanical flow-induced intracellular calcium level changed, these immortalized cells were exposed to 6.24 dynes/cm² pulsatile laminar fluid flow. On fluid stimulation, an immediate rise in intracellular calcium was detected throughout the wild type Pkd^{1+/+} cell population, peaking roughly 10-20 s after stimulation (FIGS. 9C and 9D). The [Ca²⁺]ᵢ levels then rapidly decreased but were maintained at moderate levels for 50-60 s before returning to baseline. In contrast, Pkd1-deficient osteoblasts exposed to an identical flow stimulus, an intermediate calcium response curve in the heterozygous cells and little or no calcium influx in either the early or late phase in the homozygous osteoblasts was observed. (FIGS. 9C and 9D). However, 10 mM caffeine still resulted in normal calcium influx in Pkd1 null cells after flow stimulus (data not shown), indicating the viability of the Pkd1^{null/null} and Pkd1^{m1Bei/m1Bei} cells in the loading chamber. This data suggest that the flow-induced [Ca²⁺]ᵢ response requires polycystin complex in osteoblasts and loss of Pkd1 to abolish fluid flow sensing in osteoblasts.

Referring to FIG. 9. The effects of Pkd1 deletion and mutation on baseline and flow-induced intracellular calcium ([Ca²⁺]ᵢ) response in osteoblasts as shown. A gene dose-dependent reduction of basal [Ca²⁺]ᵢ level was observed in cultured heterozygous Pkd1^{null/+} and homozygous Pkd1^{null/null} cells (n=32) (A) as well as heterozygous Pkd^{1m1Bei/+} and homozygous Pkd1^{m1Bei/m1Bei} cells (n=32) (B) compared with wild type Pkd^{1+/+} cells (n=32). Flow-induced [Ca²⁺]ᵢ responses are also impaired in a gene dose-dependent fashion in cultured heterozygous Pkd1^{null/+} and homozygous Pkd1^{null/null} cells (n=10) (C) as well as heterozygous Pkd^{m1Bei/+} and homozygous Pkd1^{m1Bei/m1Bei} cells (n=10) (D) compared with wild type Pkd^{1+/+} (n=10) cells. The immortalized osteoblasts from newborn wild type Pkd1^{+/+}, heterozygous Pkd1^{null/+} and Pkd1^{m1Bei/+}, homozygous Pkd1^{null/null} and Pkd1^{m1Bei/m1Bei} mice were cultured on type I rat tail collagen-coated 40-mm diameter glass slides at 80-90% confluency. The cells were loaded with 3 μM Fura2-AM, and the slide was then placed in an FCS2 parallel plate flow chamber. A real-time record of fluorescence intensity (F340/F380 ratio) was performed in the cells when exposed to 6.24 dynes/cm² pulsatile laminar fluid flow. The 340/380 ratios were converted to concentration using standard calibration curve. Data represent the mean±SD from these individual cells.

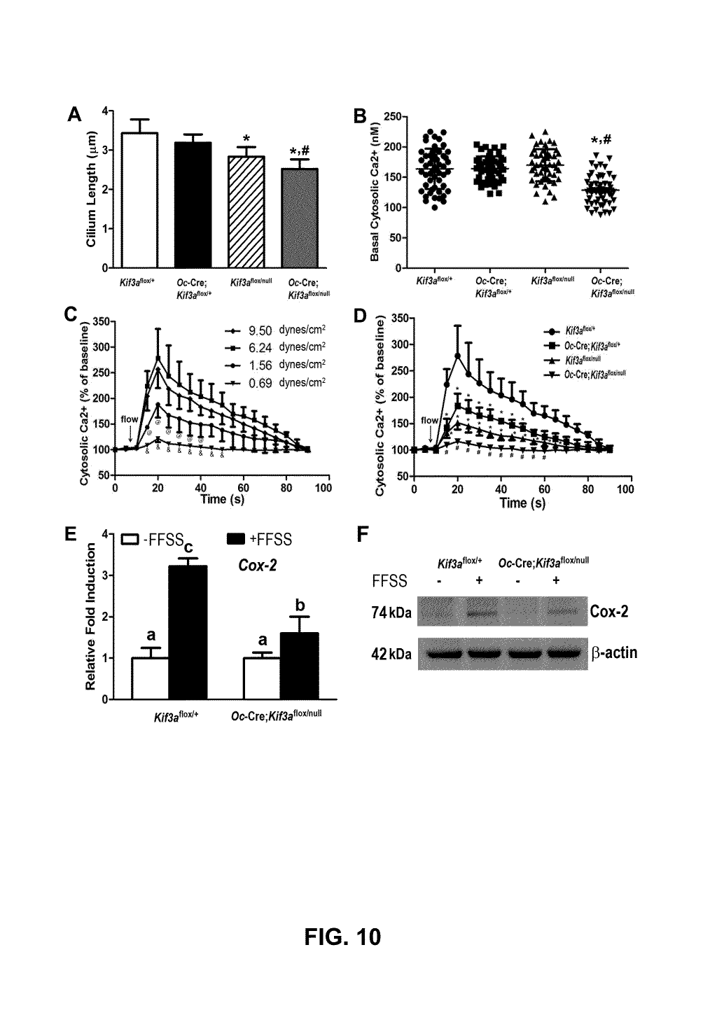

Loss of Kif3a in a gene-dose dependent fashion resulted in a reduction in the length of primary cilia in primary cultured osteoblasts and altered responses to flow shear stress, consistent with alterations of cilia functions. The length of primary cilia was reduced by approximately 27% in Kif3a^{Oc-cKO} null osteoblasts (FIG. 10A). In addition, we found that Kif3a^{Oc-cKO} null osteoblasts had a significantly lower basal intracellular calcium ([Ca²⁺]ᵢ) concentration compared with control Kif3a^{flox/+} cells; whereas single heterozygous Oc-Cre; Kif3a^{flox/+} or Kif3a^{flox/null} osteoblasts had no alterations of basal [Ca²⁺]ᵢ levels (FIG. 10B). The change in intracellular calcium was proportionate to the magnitude of the fluid flow shear stress (FFSS, 0.69˜9.5 dynes/cm²) in control Kif3a^{flox/+} osteoblasts (FIG. 10C), with 6.24 dynes/cm² achieving the highest peak flow-induced intracellular calcium response. No further significant increase in intracellular calcium was observed with 9.5 dynes/cm², indicating that 6.24 dynes/cm² is the optimal flow fluid shear stress (FFSS) to induce intracellular calcium responses in immortalized osteoblasts under these experimental conditions. Applying this amount of flow fluid shear stress (FFSS) to heterozygous Oc-Cre; Kif3a^{flox/+}, Kif3a^{flox/null} and Kif3a^{Oc-cKO} null osteoblasts demonstrated a gene-dose dependent reduction in flow-induced intracellular calcium response compared to controls (FIG. 10D), indicating that flow fluid shear stress (FFSS) is a more sensitive measurement of primary cilia abnormalities than the assessment of cilia length or number. Indeed, an intermediate calcium response curve was observed in single heterozygous cells whereas minimal calcium influx was observed in conditional Kif3a^{Oc-cKO}-null osteoblasts in response to FFSS (FIG. 10D). However, 10 mM caffeine resulted in normal calcium influx in conditional Kif3a^{Oc-cKO}-null cells after flow stimulus (data not shown), indicating the viability of the conditional Kif3a^{Oc-cKO}-null cells. Finally, the effects of flow fluid shear stress (FFSS) on mechanosensing gene expression by real-time RT-PCR and protein expression by Western blot analysis in RNA and cytoplasmic proteins isolated from control and Kif3a^{Oc-cKO}-null cells with or without flow fluid shear stress (FFSS) was studied. flow fluid shear stress (FFSS) increased both the message RNA and protein levels of Cox-2, a mechanoresponsive gene, in control osteoblasts (FIGS. 10E and 10F), whereas these changes were attenuated in Kif3a^{Oc-cKO}-null cells exposed to identical FFSS (FIGS. 10E and 10F).

Now referring to FIG. 10 the effects of global and/or Oc-Cre-mediated conditional deletion of Kif3a on baseline and flow-induced intracellular calcium ([Ca²⁺]ᵢ) response, as well as mechanoresponsive gene expression in osteoblasts as studied. (A) Length of primary cilia (n=15˜20). A gene dose-dependent reduction of cilia length was observed in the Kif3a-deficient osteoblasts compared with control Kif3a^{flox/+} cells. (B) Basal [Ca²⁺]ᵢ levels. Only Kif3a^{Oc-cKO}-null osteoblasts (n=53) showed a significantly lower basal [Ca²⁺]ᵢ levels compared with the other three group of osteoblasts (n=53). (C) Flow-induced [Ca²⁺]ᵢ response curve with different magnitudes. Control Kif3a^{flox/+} osteoblasts exhibited an flow fluid shear stress (FFSS)-dependent response of intracellular calcium. Within the four regimes of flow rates (n=4), 6.24 dynes/cm² is the optimal flow fluid shear stress (FFSS) to induce response of intracellular calcium in the immortalized osteoblasts. There was no significant difference between 6.24 dynes/cm² and 9.50 dynes/cm² loading. (D) Flow-induced [Ca²⁺]ᵢ response curve with different genotypes (n=4). A gene dose-dependent reduction of flow-induced [Ca²⁺]ᵢ responses was observed in the Kif3a-deficient osteoblasts compared with control Kif3a^{flox/+} cells, which is in agreement with the length of primary cilia in Kif3a-deficient osteoblasts. There was no significant difference between Oc-Cre; Kif3a^{flox/+} and Kif3a^{flox/null} cells.

A real-time RT-PCR and western blot analyses from control Kif3a^{flox/+} and Kif3a^{Oc-cKO}-null osteoblasts showed that mRNA and protein levels of Cox-2 were markedly increased in the loaded control cells but much less changed in the loaded Kif3a^{Oc-cKO}-null cells. Data are mean±S.D. from triple independent experiments. * significant difference from control Kif3a^{flox/+} cells at P<0.05, and # significant difference from Oc-Cre; Kif3a^{flox/+} or Kif3a^{flox/null} cells at P<0.05. @ significant difference from 9.50 dynes/cm² and 6.24 dynes/cm² at P<0.05, and & significant difference from 1.56 dynes/cm² at P<0.05. Values sharing the same superscript are not significantly different, P<0.05.

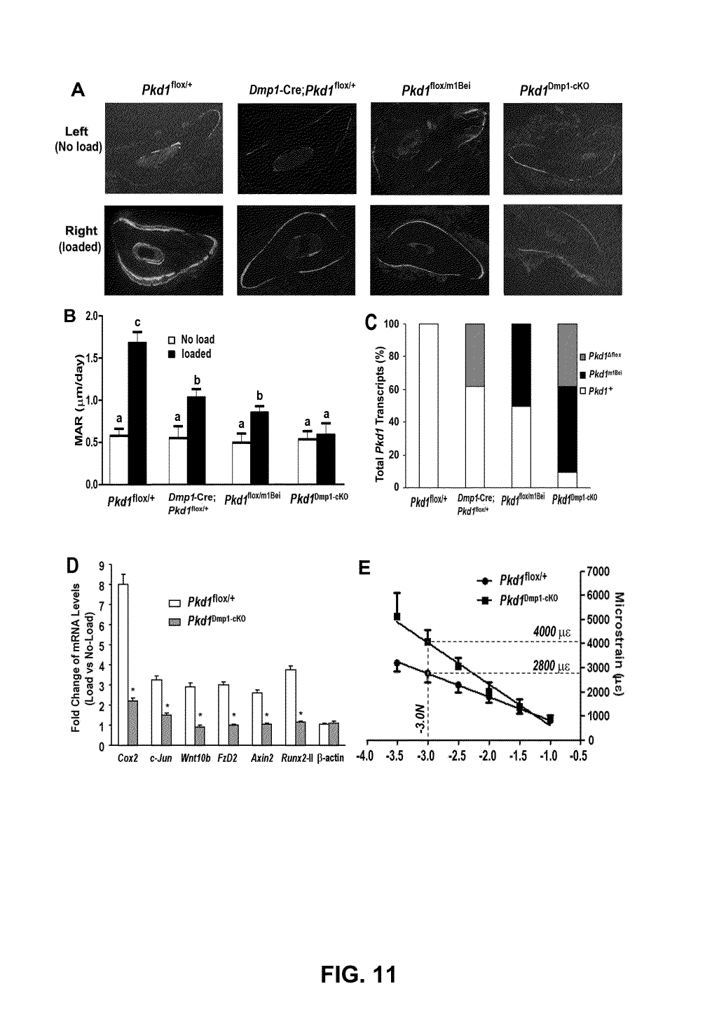

To investigate whether Pkd1 has a mechanosensing function in bone, in vivo ulnae loading experiments using 16-week-old control, single Pkd1^{flox/m1Bei} heterozygous mice, single Dmp1-Cre; Pkd^{flox/+} heterozygous mice, and conditional Pkd1^{Dmp1-cKO} null mice was performed. The same strain to the control, single Pkd1^{flox/m1Bei} heterozygous mice, single Dmp1-Cre; Pkd^{flox/+} heterozygous mice, and conditional Pkd1^{Dmp1-cKO} null mice was applied. In contrast to difference in mineral apposition rates among control, single Pkd1 heterozygous mice, and Pkd1^{Dmp1-cKO} mice at 6 weeks of age, a difference in baseline mineral apposition rate of cortical bone as assessed by calcein double labeling in the no load ulnae of control among four groups was not observed (FIG. 11A).

A robust bone formation response in the loaded ulna of control mice (FIG. 11A) was observed. In contrast, the bone formation response was markedly attenuated in the loaded ulna from these Pkd1 deficient mice (FIG. 11A). In this regard, load induced a 3-fold increase in mineral apposition rate (MAR) in the loaded ulnae from control mice, a reduction in ulna loading response was proportionate to the gene dose in single Pkd1 heterozygous and conditional Pkd1 null mice (FIG. 11B). Similar to the observation in 6-week-old mice, no load ulnae of 16-week-old Pkd1^{flox/m1Bei} and Dmp1-Cre; Pkd1^{flox/m1Bei} mice expressed 50% Pkd^{1m1Bei} mutant (functional null) allele, whereas Dmp1-Cre; Pkd1^{flox/+} and Dmp1-Cre; Pkd1^{flox/m1Bei} mice exhibited approximately 42% excision of the floxed exon 2-4 from Pkd1. Again, a net reduction of Pkd1 expression in Pkd1^{Dmp1-cKO} mice was more than 90% in no load ulnae bone samples (FIG. 11C). To examine the expression of mechanical load responsive genes in vivo, we performed real-time RT-PCR using a total of RNAs from no load and loaded ulnae of control and Pkd1^{Dmp1-cKO} mice. Consistent with the known anabolic response using this loading method, we found that Runx2-II, Cox-2, c-Jun, and Wnt-related genes (such as Wnt10b, FzD2, and Axin2) were significantly increased in the loaded ulnae from control mice (FIG. 11D), whereas these transcripts were no longer responsive to loading in Pkd1^{Dmp1-cKO} mice (FIG. 11D). To determine whether the applied stress was similar between the two groups, an ulna strain gage testing from control and conditional Pkd1^{Dmp1-cKO} mice at 16 weeks of age was performed. A linear relationship between peak compressive force (N) and peak tension microstrain (με) at the lateral ulna midshaft during cyclic axial compression loading ex vivo was observed from control Pkd1^{flox/+} and conditional Pkd1^{Dmp1-cKO} mice. It was found that ulnae from conditional Pkd1^{Dmp1-cKO} null mice had almost 1.5-fold higher microstrains at −3.0 N load compared with control mice (FIG. 11E), indicating that the attenuated anabolic response occurred in spite of increased bone stress to mechanical loading in Pkd1^{Dmp1-cKO}.

Referring to FIG. 11, conditional deletion and mutation of Pkd1 in osteocytes impairs anabolic response to mechanical loading is shown. (A) Representative images of midshaft ulnar cross-sections from no load and loaded ulnae of male control Pkd1^{flox/+} mice, single Pkd1^{flox/m1Bei} heterozygous mice, single Dmp1-Cre; Pkd1^{flox/+} heterozygous mice, and conditional Pkd1^{Dmp1-cKO} null mice after loading. There was a robust bone formation response on the medial (inset) and lateral surfaces of loaded control Pkd^{flox/+} ulna. An intermediate bone formation response was observed in the loaded ulnae of single Pkd1^{flox/m1Bei} and Dmp1-Cre; Pkd^{flox/+} heterozygous mice, but almost no response can be observed in the loaded conditional Pkd1^{Dmp1-cKO} null ulnae. (B) Graphs depicting mineral apposition rate (MAR) on periosteal surface of the midshaft ulna in response to applied mechanical strain in four genotypes. A Pkd1 gene-dose dependent impairment on MAR was observed in response to −3.0 N loading strains. (C) A real-time RT-PCR analysis of total Pkd1 transcripts in the no load ulnae of control and Pkd1-deficient mice at 16 weeks of age. Expression of total Pkd1 transcripts was performed using Pkd1-allele-specific primers as described in Experimental Procedures. Similar to the observation in 6-week-old mice, a reduction in total functional Pkd1 transcripts was proportionate to the gene dose in single Pkd1 heterozygous and conditional Pkd1 null mice. (D) Expression of mechanical load responsive genes in control and conditional Pkd1^{Dmp1-cKO} null mice 4 h after loading. A real-time RT-PCR analysis from both male genotypes shows that mRNA levels of Runx2-II, Cox-2, c-Jun, and Wnt-related genes (such as Wnt10b, FzD2, and Axin2) were significantly increased in the loaded ulnae from control Pkd^{flox/+} mice but much less change in the loaded ulnae from conditional Pkd1^{Dmp1-cKO} null mice. (E) A measurement of ulna strain gage. A linear relationship between peak compressive force (N) and peak tension microstrain (με) at the lateral ulna midshaft during cyclic axial compression loading ex vivo was observed from control Pkd1^{flox/+} and conditional Pkd1^{Dmp1-cKO} null mice. This relationship was significantly different between Pkd1^{flox/+} and Pkd1^{Dmp1-cKO} ulnae using two-way ANOVA analysis, P<0.0001. Ulnae were loaded in vivo using a peak compressive force of −3.0 N, the corresponding microstrain was 1.5-fold higher in Pkd1^{Dmp1-cKO} ulnae than in control Pkd1^{flox/+} ulnae. Data represent the mean±SD from five to six individual mice. * Indicates significant difference from control (Pkd1^{flox/+}) at P<0.05, respectively.

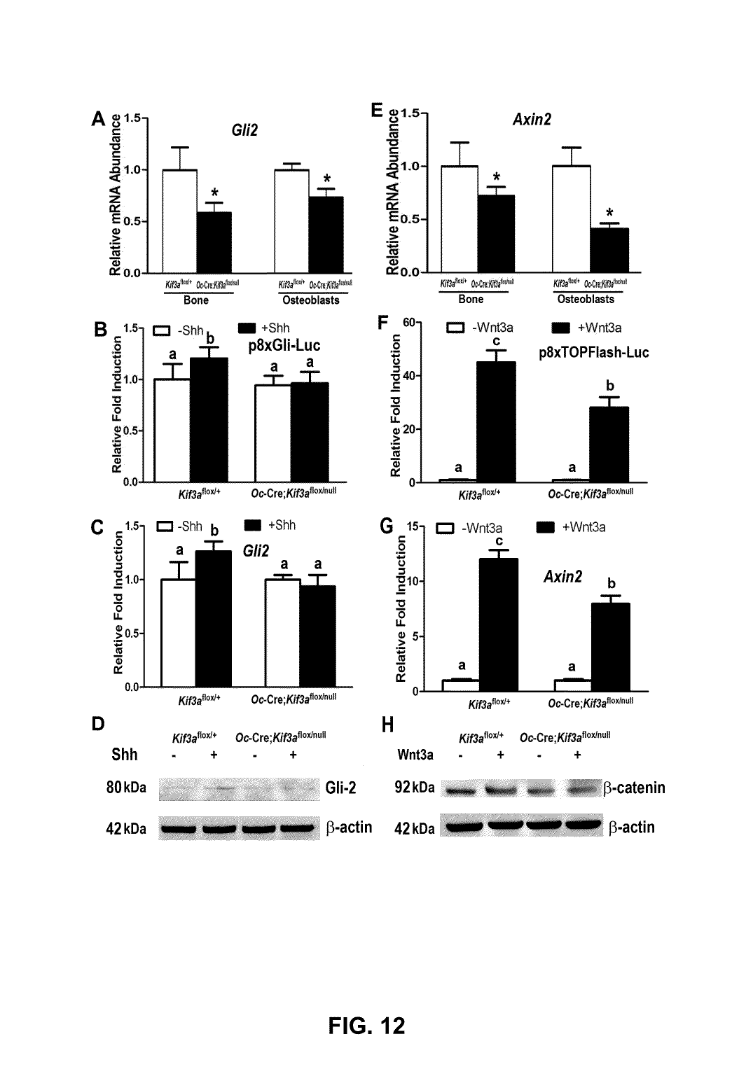

To explore additional mechanisms whereby loss of Kif3a leads to alterations in osteoblast functions, we examined other primary cilia associated signaling molecules, including patched (PTCH1)-smoothened (SMO)-hedgehog (HH)/Gli and Wnt/β-catenin pathways. Using total RNA from Kif3a^{Oc-cKO}-null tibias and cultured osteoblasts, we found that expression of Gli2, a down-stream gene of Hh signaling, was significantly decreased in Kif3a^{Oc-cKO} derived bone samples compared to Kif3a^{flox/+} controls (FIG. 12A). Addition of sonic hedgehog (Shh, 1 μg/ml) to immortalized osteoblast cultures also resulted in significant increases in Gli-responsive-promoter-luciferase activity, Gli2 mRNA expression and Gli2 protein levels in Kif3a^{flox/+} control cells (FIG. 12B-12D). In contrast, Shh failed to stimulate any of these parameters in the Kif3a^{Oc-cKO}-null osteoblasts (FIG. 12B-12D), suggesting that either loss of Kif3a and/or disruption of primary cilia function impairs Hh signaling in osteoblasts.

To examine the effect conditional deletion of Kif3a on the Wnt/β-catenin pathway, we examined the expression of Axin2, a direct down-stream gene of Wnt/β-catenin in bone and immortalized osteoblasts. We found that Axin2 was significantly lower in Kif3a^{Oc-cKO} null bone and osteoblasts compared with Kif3a^{flox/+} controls (FIG. 12E). Wnt3a/β-catenin transcriptional activity was also reduced by approximately 35% in Kif3a^{Oc-cKO}-null osteoblasts. In this regard, Wnt3a-induced a respective 45- and 12-fold increase in Super 8×TOPFlash promoter luciferase activity and Axin2 expression in control osteoblasts (FIGS. 12F and 12G), but only 28- and 8-fold increase in these parameters in Kif3a^{Oc-cKO}-null osteoblasts (FIGS. 12F and 12G). In agreement with the changes of Wnt/β-catenin downstream signaling above, Wnt3a-induced accumulation of cytoplasmic β-catenin protein was reduced in Kif3a^{Oc-cKO}-null osteoblasts compared to controls (FIG. 6H), indicating that conditional deletion of Kif3a significantly attenuates Wnt/β-catenin signaling in osteoblasts.

Now referring to FIG. 12 the effects of global and/or Oc-Cre-mediated conditional deletion of Kif3a on Hh and Wnt signaling in bone and osteoblasts was examined. (A-D) Hh/Gli2 pathway. Both tibias and cultured osteoblasts exhibited significant downregulation of Gli2 mRNA messages in the Kif3a^{Oc-cKO} group compared to Kif3a^{flox/+} controls. An administration of Shh (1 μg/ml) resulted in significant increases in 8×Gli-responsive luciferase (p8×Gli-Luc) activity and mRNA and protein levels of Gli2 in Kif3a^{flox/+} control cells, whereas less or no stimulation with Shh was observed in the Kif3a^{Oc-cKO}-null osteoblasts. (D-H) Wnt/β-catenin pathway. Both tibias and cultured osteoblasts showed significant lower mRNA expression of Axin2 in the Kif3a^{Oc-cKO} group compared with Kif3a^{flox/+} controls. Wnt3a-induced Super 8×TOPFlash luciferase (p8×TOPFlash-Luc) activity, the level of cytoplasmic β-catenin protein, and Axin2 mRNA expression were much higher in the control cells than in the Kif3a^{Oc-cKO}-null osteoblasts. Data represent the mean±S.D. from five to six individual mice in triple independent experiments. *significant difference from control (Kif3a^{flox/+}), P<0.05, respectively. Values sharing the same superscript are not significantly different, P<0.05.

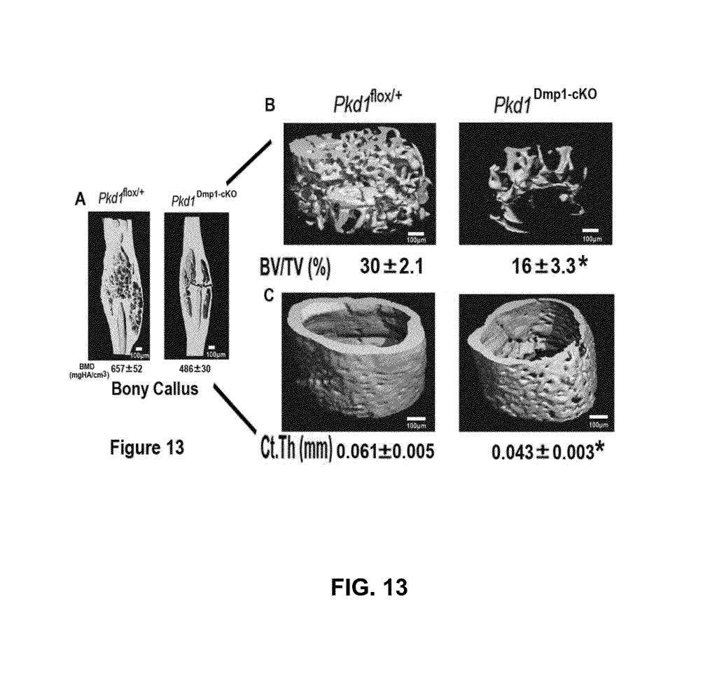

Now referring to FIG. 13 that shows modulation of fracture healing by conditional deletion of Pkd1 in bone. To investigate whether Pkd1 has a role in fracture healing process, we created in vivo fibula transverse fracture mouse model by osteotomy using 12-week-old control and conditional Pkd1^{Dmp1-cKO} null mice. We observed a much small bony callus in Pkd1^{Dmp1-cKO} mice (FIG. 13 A). A 26% reduction of bone mineral density (mgHA/cm3), 657±52 vs 486±30) in bony callus was observed in Pkd1^{Dmp1-cKO} mice compared with age-matched control mice (Pkd1^{flox/+}) (FIG. 13 A). CT analysis revealed that the lower bone mass in Pkd1^{Dmp1-cKO} mice caused by a respective 47% and 30% reduction in trabecular bone volume [BV/TV (%), 16±3.3 vs 30±2.1](FIG. 13B) and cortical bone thickness [CtTh (mm), 0.043±0.003 vs 0.061±0.005] (FIG. 13C) compared with control Pkd1^{flox/+} mice, indicating that the conditional deletion of Pkd1 in osteocytes in bone resulted in a significant delay in bone fracture healing. These findings suggest that activation of Pkd1 signaling could modulate bone fracture healing process in clinical applications.

### Example 1

Mice:

Dentin matrix protein 1 (Dmp1)-Cre mice (Lu, Y et al. (2007) DMP1-targeted Cre expression in odontoblasts and osteocytes. J. Dent. Res. 86, 320-325) were crossed with floxed Pkd1 mice obtained from Dr. Gregory Germino at Johns Hopkins University (Piontek, K. B., et al., (2004) A functional floxed allele of Pkd1 that can be conditionally inactivated in vivo. J. Am. Soc. Nephrol. 15, 3035-3043). The Pkd1^{m1Bei} heterozygous mice were available in our laboratory as previously described (Xiao, Z et al., (2006) Cilia-like structures and polycystin-1 in osteoblasts/osteocytes and associated abnormalities in skeletogenesis and Runx2 expression. J. Biol. Chem. 281, 30884-30895). These mice were bred and maintained on a C57BL/6J background. Because Cre-recombinase-mediated deletion of single flox/m1Bei (which functions as a null allele) allele reduces the risk of mosaicism that may occur because of the less than 100% efficiency of Cre-recombinase to excise two floxed alleles (flox/flox) (Kwan, K. M. (2002) Conditional alleles in mice: practical considerations for tissue-specific knockouts. Genesis 32, 49-62) we created double heterozygous Dmp1-Cre; Pkd^{1m1Bei/+} mice and homozygous Pkd1^{flox/flox} mice. Double heterozygous Dmp1-Cre; Pkd^{1m1Bei/+} mice were mated with homozygous Pkd^{flox/flox} mice to generate excised floxed Pkd1 heterozygous (Dmp1-Cre; Pkd^{flox/+}) and null mice (Dmp1-Cre; Pkd1^{flox/m1Bei} or Pkd1^{Dmp1-cko}), as well as Beier Pkd1 heterozygous mice (Pkd1^{m1Bei/flox}) and Dmp1-Cre negative control mice (Pkd1^{flox/+}, equivalent to wild type). These mice were used for skeletal phenotype analysis and mechanical loading experiments. All animal research was conducted according to guidelines provided by the National Institutes of Health and the Institute of Laboratory Animal Resources, National Research Council. The University of Tennessee Health Science Center (protocol number 1889) and University of Kansas Medical Center's (protocol number 2007-1630) Animal Care and Use Committee approved all animal studies.

Genotyping Polymerase Chain Reaction (PCR) and Real-Time PCR to Detect Mutations and Deletions:

Genomic DNA was prepared from tail and other tissue specimens by using standard procedures (Xiao, Z et al. (2005) Selective Runx2-II deficiency leads to low-turnover osteopenia in adult mice. Dev. Biol. 283, 345-356) PCR genotyping was performed using the following primers (Piontek, K. B, et al., (2004) A functional floxed allele of Pkd1 that can be conditionally inactivated in vivo. J. Am. Soc. Nephrol. 15, 3035-3043) (FIG. 1A): F1, 5′-CTT CTA TCG CCT TCT TGA CGA GTT C-3′ (SEQ ID NO:1); R1, 5′-AGG GCT TTT CTT GCT GGT CT-3′ (SEQ: ID NO:2) and R2,5′-TCG TGT TCC CTT ACC AAC CCT C-3′ (SEQ: ID NO:3). Pkd1 floxed (Pkd1^{flox}) alleles were identified in 2% agarose gels as 670-bp bands (FIG. 1B). The delta floxed Pkd1 (Pkd1^{Δflox}) allele was detected as a 0.85-kb band in 1% agarose gels (FIG. 1B) (Xiao, Z., et al. (2010) Conditional disruption of Pkd1 in osteoblasts results in osteopenia due to direct impairment of bone formation. J. Biol. Chem. 285, 1177-1187). The Pkd1^{m1Bei} allele was genotyped using SYBR Green (Bio-Rad, Hercules, Calif.) real-time PCR as previously described (Xiao, Z et al., (2006) Cilia-like structures and polycystin-1 in osteoblasts/osteocytes and associated abnormalities in skeletogenesis and Runx2 expression. J. Biol. Chem. 281, 30884-30895)

Bone mineral density (BMD) of femurs was assessed at 6 and 16 weeks of age with a LUNAR_{PIXIMUS} bone densitometer (Lunar Corp., Madison, Wis., USA). Calcein (Sigma, St. Louis, Mo., USA) double labeling of bone and histomorphometric analyses of periosteal mineral apposition rate (MAR) in tibias were performed using the osteomeasure analysis system (Osteometrics). (Glass, D. A., et al. (2005) Canonical Wnt signaling in differentiated osteoblasts controls osteoclast differentiation. Dev. Cell 8, 751-764). The distal femoral metaphyses were also scanned with a Scanco μCT 40 (Scanco Medical AG, Brüttisellen, Switzerland). 3D-images were analyzed to determine bone volume/trabecular volume (BV/TV) and cortical thickness (Ct.Th) as previously described (Xiao, Z et al., (2005) Selective Runx2-II deficiency leads to low-turnover osteopenia in adult mice. Dev. Biol. 283, 345-356).

On the day of testing, the femurs were thawed and rehydrated with 1×PBS for 3 h. Bending tests were performed using a three-point fixture on an electromechanical testing system (ELF 3200, EnduraTEC, Inc., Minnetonka, Minn., USA). The bones were flexed in the anterior-posterior plane by displacing the loading point fixture at 5 mm/min until failure (66). Bending force-deflection curves were constructed and analyzed for stiffness (S), maximum force to failure (Fₘₐₓ). The flexural rigidity (EI) was calculated from the classical equations of beam theory for a simply-supported beam with a central concentrated load.

Serum Biochemistry:

Serum osteocalcin levels were measured using a mouse osteocalcin EIA kit (Biomedical Technologies Inc., Stoughton, Mass., USA). Serum urea nitrogen (BUN) was determined using a BUN diagnostic kit from Pointe Scientific, Inc. Serum calcium (Ca) was measured by the colorimetric cresolphthalein binding method, and phosphorus (P) was measured by the phosphomolybdate-ascorbic acid method (Stanbio Laboratory, TX, USA). Serum osteoprotegerin (OPG) and Rank ligand (Rank-L) were measured using mouse ELISA kits (Quantikine, R&D Systems), and serum tartrate-resistant acid phosphatase (TRAP) was assayed with the ELISA-based SBA Sciences mouse TRAP™ assay (Immunodiagnostic Systems, Fountain Hills, Ariz., USA).

Calvaria from wild type (Pkd1^{+/+}), heterozygous Pkd1^{null/+} and Pkd1^{m1Bei/+}, and homozygous Pkd1^{null/null} and Pkd1^{m1Bei/m1Bei} embryos at embryonic day 15.5 (E15.5) were used to isolate osteoblasts by sequential collagenase digestion. To engineer immortal osteoblast cell lines, isolated primary osteoblasts were infected using a retroviral vector carrying SV40 large and small T antigen as previously described (Xiao, Z., et al. (2006) Cilia-like structures and polycystin-1 in osteoblasts/osteocytes and associated abnormalities in skeletogenesis and Runx2 expression. J. Biol. Chem. 281, 30884-30895). For flow experiments, the immortalized cells were cultured on type I rat tail collagen-coated 40-mm diameter glass slides at 80-90% confluency in alpha-minimal essential medium (c-MEM) containing 2% fetal bovine serum (FBS) and 1% penicillin and streptomycin (P/S) for 3 days. Cells were rinsed two times with Hanks' balanced saline solution (HBSS). The cells were then loaded with 3 μM Fura2-AM (Molecular Probes, Eugene, Oreg., USA), a fluorescent Ca²⁺ probe, in HBSS for 30 min at 37° C. Loaded cells were incubated for an additional 45 min with HBSS alone to ensure complete deesterification of the fluorescent molecule. A glass slide was then placed in an FCS2 parallel plate flow chamber (Bioptechs, Inc., Butler, Pa., USA), 0.25×14×22 mm. A fresh bolus of flow media was added to the chamber, and the cells were left undisturbed for 30 min. The flow media consisted of phenol-free c-MEM and 2% FBS equilibrated with 5% CO₂/95% air at 37° C. The chamber was mounted on the stage of an inverted microscope with CCD camera to allow real-time record of fluorescence intensity (F340/F380 ratio) to generate ratiometric video images of individual static cells or cells exposed to flow (Intracellular Imaging, Inc., Cincinnati, Ohio, USA). The F340/F380 ratios were converted to concentration using standard calibration curve. Baseline levels of [Ca²⁺]ᵢ were obtained for 3 min before exposing to flow. The slides of cells were exposed to 6.24 dynes/cm² pulsatile laminar fluid flow for 3 min with a peristaltic pump (Bioptechs, Inc., Butler, Pa., USA). A ratiometric video image analysis at individual cells was used to determine changes in [Ca²⁺]ᵢ.

The forearm compression ulna loading was performed starting at 16 weeks of age in male control Pkd1^{flox/+} mice, single Pkd1^{flox/m1Bei} heterozygous mice, single Dmp1-Cre; Pkd1^{flox/+} heterozygous mice, and Dmp1-Cre; Pkd1^{flox/m1Bei} conditional null mice as previously described (Li, J., et al., (2005) The P2X7 nucleotide receptor mediates skeletal mechanotransduction. J. Biol. Chem. 280, 42952-42959) (Robling, A. G., et al., (2003) Evidence for a skeletal mechanosensitivity gene on mouse chromosome 4. FASEB J. 17, 324-326). Briefly, each animal was anesthetized with 4% isofluorane inhalation, and the right forearm was positioned between two brass cups and loaded in compression with a mechanical testing machine (Bose ElectroForce 3200, Eden Prairie, Minn., USA). The right ulnae were loaded at −3.0 N, haversine waveform, 2 Hz, 180 cycles, on Mondays, Wednesdays, and Fridays for 2 weeks. To measure strain-induced new bone formation, calcein (20 mg/kg) was injected into the mice 12 and 3 days before sacrifice. The ulnae and radii were removed for further analysis of bone formation rate and bone histomorphometry. The femurs were dissected and cleaned from muscle and soft tissue attachments and then frozen at −20° C. for biomechanical property testing.

For quantitative real-time RT-PCR, 2.0 g total RNA isolated from whole tibias of 6-week-old mice, no load ulnae of 16-week-old mice, whole loaded and no load ulnae of 18-week-old mice, and 10-day cultured primary osteoblasts in differentiation media were reverse transcribed as previously described (72). PCR reactions contained 100 ηg template (cDNA or RNA), 300 ηM each forward and reverse primers, and 1×iQ™ SYBR® Green Supermix (Bio-Rad, Hercules, Calif., USA) in 50 μl. The threshold cycle (Ct) of tested gene product from the indicated genotype was normalized to the Ct for cyclophilin A. Expression of total Pkd1 transcripts was performed using the following Pkd1-allele-specific primers: In exon 26, forward primer of normal Pkd¹⁺ transcript: 5′-CTG GTG ACC TAT GTG GTC AT-3′ (SEQ: ID NO:4), forward primer of mutant Pkd^{1m1Bei} transcript: 5′-CTG GTG ACC TAT GTG GTC AG-3′ (SEQ: ID NO:5), and common reverse primer: 5′-AGC CGG TCT TAA CAA GTA TTT C-3′ (SEQ: ID NO:6); in exon 2-4, forward primer of normal Pkd¹⁺ transcript: 5′-ATA GGG CTC CTG GTG AAC CT-3′ (SEQ: ID NO:7) and reverse primer: 5′-CCA CAG TTG CAC TCA AAT GG-3′ (SEQ: ID NO:8). The normal Pkd¹⁺ vs. cyclophilin A was normalized to the mean ratio of five control mice, which was set to 1. The percentage of conditional deleted and mutant transcripts was calculated from the relative levels of the normal Pkd¹⁺ transcripts in different Pkd1 exons (Xiao, Y, et al. (2010) Overexpression of Trpp5 contributes to cell proliferation and apoptosis probably through involving calcium homeostasis. Mol. Cell. Biochem. 339, 155-161) All primer sequences of other genes used in real-time RT-PCR were provided in Table 7.

TABLE 7
Primer sequences used in real-time RT-PCT

| | Accession | SEQUENCE ID | Forward primer |
|---|---|---|---|
| Gene | no. | NUMBER | |
| Runx2-II | NM_009820 | SEQ ID 9 | 5′-GCCTCACAAACAACCACAGA-3′ |
| Runx2-I | D14636 | SEQ ID 10 | 5′-TCGCTAACTTGTGGCTGTTG-3′ |
| Runx2 | NM_009820 | SEQ ID 11 | 5′-TCTGGCCTTCCTCTCTCAG-3′ |
| Osteocalcin | NM_007541 | SEQ ID 12 | 5′-AGCAGGAGGGCAATAAGGTA-3′ |
| Osteopontin | AF515708 | SEQ ID 13 | 5′-TCTGATGAGACCGTCACTGC-3′ |
| Bsp | NM_008318 | SEQ ID 14 | 5′-GCCTCAGTTGAATAAACATGAAA-3′ |
| Opg | MMU94331 | SEQ ID 15 | 5′-GTTCCTGCACAGCTTCACAA-3′ |
| Rank ligand | NM_011613 | SEQ ID 16 | 5′-GCAGAAGGAACTGCAACACA-3′ |
| Mmp13 | NM_008607 | SEQ ID 17 | 5′-AGTTGACAGGCTCCGAGAAA-3′ |
| Dmp1 | MMU242625 | SEQ ID 18 | 5′-AGTGAGGAGGACAGCCTGAA-3′ |
| Phex | NM_011077 | SEQ ID 19 | 5′-CGCCTGACAAACTTTTGAGACC-3′ |
| Sost | NM_024449 | SEQ ID 20 | 5′-AAGCCGGTCACCGAGTTGGT-3′ |
| Trap | NM_007388 | SEQ ID 21 | 5′-AACACCACGAGAGTCCTGCT-3′ |
| Mmp9 | NM_013599 | SEQ ID 22 | 5′-AGTTGCCCCTACTGGAAGGT-3′ |
| Collagen II | NM_031163 | SEQ ID 23 | 5′-TGGCTTCCACTTCAGCTATG-3′ |
| VegfA | NM_009505 | SEQ ID 24 | 5′-GGGTGCACTGGACCCTGGGTTTAC-3′ |
| PPARγ | NM_009505 | SEQ ID 25 | 5′-CAAGGTGCTCCAGAAGATGA-3′ |
| aP2 | NM_024406 | SEQ ID 26 | 5′-GCGTGGAATTCGATGAAATCA-3′ |
| Cyclophilin A | NM_008907 | SEQ ID 27 | 5′-CTGCACTGCCAAGACTGAAT-3′ |
| | Accession | SEQUENCE ID | |
| Gene | no. | NUMBER | Reverse primer |
| Runx2-II | NM_009820 | SEQ ID 28 | 5′-TTAAACGCCAGAGCCTTCTT-3′ |
| Runx2-I | D14636 | SEQ ID 29 | 5′-GCTCACGTCGCTCATCTTG-3′ |
| Runx2 | NM_009820 | SEQ ID 30 | 5′-GGATGAAATGCTTGGGAAC-3′ |
| Osteocalcin | NM_007541 | SEQ ID 31 | 5′-CAAGCAGGGTTAAGCTCACA-3′ |
| Osteopontin | AF515708 | SEQ ID 32 | 5′-CCTCAGTCCATAAGCCAAGC-3′ |
| Bsp | NM_008318 | SEQ ID 33 | 5′TCCTCACCCTTCAATTAAATCCCACAA-3 ′ |
| Opg | MMU94331 | SEQ ID 34 | 5′-AAACAGCCCAGTGACCATTC-3′ |
| Rank ligand | NM_011613 | SEQ ID 35 | 5′-TGATGGTGAGGTGTGCAAAT-3′ |
| Mmp13 | NM_008607 | SEQ ID 36 | 5′-GGCACTCCACATCTTGGTTT-3′ |
| Dmp1 | MMU242625 | SEQ ID 37 | 5′-GAGGCTCTCGTTGGACTCAC-3′ |
| Phex | NM_011077 | SEQ ID 38 | 5′-TGCTCCCTGTTTCTGCTTCC-3′ |
| Sost | NM_024449 | SEQ ID 39 | 5′-GTGAGGCGCTTGCACTTGCA-3′ |
| Trap | NM_007388 | SEQ ID 40 | 5′-GTACCAGGGCAGAGAAGCTG-3′ |
| Mmp9 | NM_013599 | SEQ ID 41 | 5′-GTGGATAGCTCGGTGGTGTT-3′ |
| Collagen II | NM_031163 | SEQ ID 42 | 5′-AGGTAGGCGATGCTGTTCTT-3′ |
| VegfA | NM_009505 | SEQ ID 43 | 5′-CCTGGCTCACCGCCTTGGCTTGTC-3′ |
| PPARγ | NM_009505 | SEQ ID 44 | 5′-AGTAGCTGCACGTGCTCTGT-3′ |
| aP2 | NM_024406 | SEQ ID 45 | 5′-CCCGCCATCTAGGGTTATGA-3′ |
| Cyclophilin A | NM_008907 | SEQ ID 46 | 5′-CCACAATGTTCATGCCTTCT-3′ |

Strain Gage Testing:

The right arms were isolated from male Pkd1^{flox/+} control and Dmp1-Cre; Pkd1^{flox/m1Bei} conditional null mice at 16 weeks of age. Loading strain was determined using a single element strain gage (EA-06-015-DJ-120, Vishay Intertechnology, Inc.) attached to the medial surface of the ulnar midshaft as previously described (Robling, A. G., et al., (2003) Evidence for a skeletal mechanosensitivity gene on mouse chromosome 4. FASEB J. 17, 324-326). After applying strain gages, loading was conducted on the intact right forearm with 1.0, 1.5, 2.0, 2.5, 3.0, and 3.5 N compressive forces in a haversine waveform at 2 Hz for 15 cycles. Strains generated from ulnae in the last five cycles were averaged.

Primary Osteoblast Culture for Proliferation and Differentiation and Gene Expression Profiles:

Primary osteoblasts were isolated from the newborn mouse calvarias by sequential collagenase digestion at 37° C. as previously described (60, 72). The cells were cultured in a-MEM containing 10% FBS and 1% P/S. Cell proliferation was detected by BrdU incorporation assays following the manufacturer's directions (QIA58, Calbiochem, Gibbstown, N.J., USA). To induce differentiation, primary osteoblasts were plated at a density of 2×10⁴ cells per well in a 12-well plate and 4×10⁴ cells per well in a 6-well plate and grown up to 21 days in α-MEM containing 10% FBS supplemented with 5 mM β-glycerophosphate and 25 μg/ml ascorbic acid. Alkaline phosphatase activity and Alizarin red-S histochemical staining for mineralization were performed as previously described (Xiao, Z. S., et al. (2004) Selective deficiency of the “bone-related” Runx2-II unexpectedly preserves osteoblast-mediated skeletogenesis. J. Biol. Chem. 279, 20307-20313). Total DNA content was measured with a PicoGreen® dsDNA quantitation reagent and kit (Molecular Probes, Eugene, Oreg., USA). Protein concentrations of the supernatant were determined with a Bio-Rad protein assay kit (Bio-Rad, Hercules, Calif., USA). For gene expression profiles, 2.0 μg of total RNA were isolated from primary osteoblasts cultured 4, 14, and 21 days in differentiation media. The cDNAs were generated using a Reverse Transcriptase Kit (Perkin-Elmer, Foster City, Calif., USA). PCR reactions contained 100 ηg template (cRNA or cDNA), 200 mmol each forward and reverse primers, 1×iQ™ SYBR® Green Supermix (Bio-Rad, Hercules, Calif., USA) in 50 μl. The Ct of tested gene product from the indicated genotype was normalized to the Ct for cyclophilin A as previously described (Xiao, Z., et al., (2008) Polycystin-1 regulates skeletogenesis through stimulation of the osteoblast-specific transcription factor RUNX₂-II. J. Biol. Chem. 283, 12624-12634)

Primary osteoblasts were grown on collagen-coated 4-well chambers at 1×10⁵ cells per well and kept at confluence for at least 3 days. At the end of the culture, the cells were washed three times with PBS, fixed with cold 4% paraformaldehyde/0.2% Triton for 10 min at room temperature, and washed with PBS three times. The cells were incubated for 30 min in 1% BSA before incubation with primary acetylated alpha-tubulin antibody (1:4000, Sigma Aldrich, T6793) for 1 h at room temperature. After washing three times in PBS, they were treated with secondary Texas Red-labeled anti-mouse IgG (Jackson ImmunoResearch, 715-076-150) in 1% BSA for 1 h at room temperature and washed three times in PBS before mounting with ProLong® Gold antifade reagent (Invitrogen, P36935). Nuclei were counterstained with DAPI blue. Photographs were taken under a microscope with magnification of 40× for counting the number of primary cilia in cultured primary osteoblasts as previously described (Xiao, Z, et al., (2006) Cilia-like structures and polycystin-1 in osteoblasts/osteocytes and associated abnormalities in skeletogenesis and Runx2 expression. J. Biol. Chem. 281, 30884-30895)

The differences between two groups by unpaired t-test and multiple groups by one-way analysis of variance were analyzed. All values are expressed as means±.D. All computations were performed using GraphPad Prism5 (GraphPad Software Inc. La Jolla, Calif., USA).

### Example 2

### Materials and Methods

The floxed Kif3a mice were obtained from Lawrence S. B. Goldstein at the University of California San Diego (Marszalek, J et al., (1999). Situs inversus and embryonic ciliary morphogenesis defects in mouse mutants lacking the KIF3A subunit of kinesin-II. Proc Natl Acad Sci U S A 96, 5043-5048) and Osteocalcin (Oc)-Cre mice from Dr. Thomas Clemens at the University of Alabama (Zhang, M. et al., (2002) J Biol Chem 277, 44005-44012). The Kif3a^{null/+} heterozygous mice were available in our laboratory as previously described (Qiu, N., et al., (2010). Kif3a deficiency reverses the skeletal abnormalities in Pkd1 deficient mice by restoring the balance between osteogenesis and adipogenesis. PLoS One 5, e15240). These mice were bred and maintained on a C57BL/6J background. Because Cre-recombinase-mediated deletion of a single flox/null allele reduces the risk of mosaicism that may occur because of the less than 100% efficiency of Cre-recombinase to excise two floxed alleles (flox/flox) (Kwan, K. M. (2002). Conditional alleles in mice: practical considerations for tissue-specific knockouts. Genesis 32, 49-62) we created double heterozygous Oc-Cre; Kif3a^{null/+} mice and homozygous Kif3a^{flox/flox} mice. Double heterozygous Oc-Cre; Kif3a^{null/+} mice were mated with homozygous Kif3a^{flox/flox} mice to generate excised floxed Kif3a heterozygous (Oc-Cre; Kif3a^{flox/+}) and null mice (Oc-Cre; Kif3a^{flox/null} or Kif3a^{Oc-cKO}), as well as Kif3a heterozygous mice (Kif3a^{null/flox}) and Oc-Cre-negative control mice (Kif3a^{flox/+}, equivalent to wild type). These last four genotype mice were used for skeletal phenotype analysis and primary osteoblast cultures. All animal research was conducted according to guidelines provided by the National Institutes of Health and the Institute of Laboratory Animal Resources, National Research Council. The University of Tennessee Health Science Center's Animal Care and Use Committee approved all animal studies (Protocol number 1885 and 1889).

Genomic DNA was prepared from tail and other tissue specimens by using standard procedures (Xiao et al., 2005). PCR genotyping was performed using the following primers (FIG. 2A) (Lin et al., 2003; Qiu et al., 2010): Kif3a wild type (Kif3a⁺) and floxed (Kif3a^{flox}) alleles, F1, 5′-AGG GCA GAC GGA AGG GTG G-3′ (SEQ: ID NO: 47) R1, 5′-TCT GTG AGT TTG TGA CCA GCC-3′; (SEQ: ID NO:48) Kif3a null (Kif3a^{null}) and conditional null (Kif3a^{Δflox}) alleles, F1, 5′-AGG GCA GAC GGA AGG GTG G-3′ (SEQ: ID NO:49), R2,5′-TGG CAG GTC AAT GGA CGC AG-3′ (SEQ: ID NO: 50) The Kif3a floxed (Kif3a^{flox}), wild type (Kif3a⁺), and Kif3a null/conditional null (Kif3a^{null} or Kif3a^{Δflox}) alleles were identified in 2% agarose gels as 490-bp, 360-bp, and 200-bp bands (FIG. 1B), respectively (Lin, F., et al., (2003). Kidney-specific inactivation of the KIF3A subunit of kinesin-II inhibits renal ciliogenesis and produces polycystic kidney disease. Proc Natl Acad Sci USA 100, 5286-5291), (Qiu, N., et al., (2010). Kif3a deficiency reverses the skeletal abnormalities in Pkd1 deficient mice by restoring the balance between osteogenesis and adipogenesis. PLoS One 5, e15240)

BMD of femurs was assessed at 6 and 24 weeks-of-age with a LUNAR_{PIXIMUS} bone densitometer (Lunar Corp., Madison, Wis., USA). Calcein (Sigma, St. Louis, Mo., USA) double labeling of bone and histomorphometric analyses of periosteal MAR in tibias were performed using the osteomeasure analysis system (OsteoMetrics, Decatur, Ga., USA) (Glass, D. A., et al. (2005). Canonical Wnt signaling in differentiated osteoblasts controls osteoclast differentiation. Dev Cell 8, 751-764.) (Xiao, Z., et al., (2005). Selective Runx2-II deficiency leads to low-turnover osteopenia in adult mice. Dev Biol 283, 345-356). The distal femoral metaphyses were also scanned with a Scanco μCT 40 (Scanco Medical AG, Brüttisellen, Switzerland). 3D-images were analyzed to determine bone volume/trabecular volume and cortical thickness as previously described (Xiao, Z., et al., (2005). Selective Runx2-II deficiency leads to low-turnover osteopenia in adult mice. Dev Biol 283, 345-356).

For each femur, μCT data (1520 slices per femur) were imported into the imaging software, Amira (Pro Medicus Limited, Richmond, Australia), and saved in a DICOM file format. Measurements of the inner and outer diameters in both the x and y directions were taken using a DICOM viewer (Santa DICOM Viewer FREE, Santesoft, Athens, Greece). To increase the accuracy of the measurements, a threshold on the luminosity was applied with a lower limit of 1,000 and an upper limit of 1,500. For each specimen, three slices were measured, and the averages were used for the calculations. From this data, the moment area of inertia (I) was calculated using the formula

$$I = \frac{\text{?}}{64} ( {X}_{1} \text{?} - {X}_{2} \text{?} ) , \\ \text{?} \text{indicates text missing or illegible when filed}$$

where X₁ and Y₁ are the outer diameters and X₂ and Y₂ are the inner diameters. After I was calculated, it was combined with the force-displacement data from the three-point bending tests to calculate the apparent elastic modulus, Eₐₚₚ, using the following expression:

$${E}_{app} = \frac{\text{?}}{\text{?}} , \\ \text{?} \text{indicates text missing or illegible when filed}$$

where F is the applied force, L is the span, and δ is the deflection. Gap Bulletin per KZ.

Eight femurs from 6-week-old male mice and eight femurs from 24-week-old male mice were acquired for the three-point bend testing. Femurs were stored at 4° C. prior to acquisition. After acquisition, the femurs were stored at 0° C. until being thawed in 1× phosphate buffered saline (PBS) 5 min prior to testing. The distance between the supports was held constant for all femurs at 6 mm, and the radius of supports was 0.5 mm. The femurs were tested using an Instron 33R (Instron, Norwood, Mass.) at a rate of 2 mm/sec to a 40% decline in maximum load. Load magnitude and displacement data were collected by Bluehill® Materials Testing Software (Instron, Norwood, Mass.).

Serum Osteocalcin levels were measured using a mouse Osteocalcin EIA kit (Biomedical Technologies, Inc., Stoughton, Mass., USA). Serum BUN was determined using a BUN diagnostic kit from Pointe Scientific, Inc (Canton, Mich., USA). Serum calcium (Ca) was measured by the colorimetric cresolphthalein binding method, and phosphorus (P) was measured by the phosphomolybdate-ascorbic acid method (Stanbio Laboratory, Boerne, Tex., USA). Serum OPG and Rank ligand (RankL) were measured using mouse ELISA kits (Quantikine®, R&D Systems, Minneapolis, Minn., USA), and serum TRAP was assayed with the ELISA-based SBA Sciences mouse TRAP™ assay (Immunodiagnostic Systems, Fountain Hills, Ariz., USA).

For quantitative real-time RT-PCR, 1.0 μg total RNA isolated from whole tibias of 6-week-old control and Kif3a-deficient mice was reverse transcribed as previously described (Xiao et al., 2004). PCR reactions contained 20 ηg template (cDNA or RNA), 375 mM each forward and reverse primers, and 1×SsoFast™ EvaGreen® supermix (Bio-Rad, Hercules, Calif., USA) in a total of 10 μl reaction volume. The threshold cycle (Ct) of tested gene product from the indicated genotype was normalized to the Ct for cyclophilin A. Expression of total Kif3a transcripts was performed using the following Kif3a-allele-specific primers in exon 2: forward primer of normal Kif3a⁺ transcript (Kif3a⁺ plus Kif3^{flox}): 5′-GCT ATA GAC AGG CCG TCA GC-3′ (SEQ: ID NO: 51) reverse primer: 5′-GTC TTT GGA GGT TCG TTG GA-3′ (SEQ: ID NO: 52) he normal Kif3a⁺ vs. cyclophilin A was normalized to the mean ratio of five control mice, which was set to 1. The percentage of Kif3a null (Kif3a^{null}) and/or conditional deleted (Kif3a^{flox}) transcripts was calculated from the relative levels of the normal Kif3a⁺ transcripts in different Kif3a-deficient mice (Xiao, Y., et al. (2009). Overexpression of Trpp5 contributes to cell proliferation and apoptosis probably through involving calcium homeostasis. Mol Cell Biochem 339, 155-161). All primer information of other genes used in real-time RT-PCR can be found in our previous report (Xiao, Z., et al., (2011). Conditional deletion of Pkd1 in osteocytes disrupts skeletal mechanosensing in mice. FASEB J 25, 2418-2432).

Calvaria from E17.5 control and Kif3a-deficient embryos were used to isolate primary osteoblasts by sequential collagenase digestion at 37° C. To engineer immortal osteoblast cell lines, isolated primary osteoblasts were infected using a retroviral vector carrying SV40 large and small T antigen as previously described (Borton, A. J., et al., (2001). The loss of Smad3 results in a lower rate of bone formation and osteopenia through dysregulation of osteoblast differentiation and apoptosis. J Bone Miner Res 16, 1754-1764), (Xiao, Z. S., et al., (2004) J Biol Chem 279, 20307-20313). Briefly, cells were grown in 100-mm plates at 50-60% confluence the day before infection. On the day of infection, the medium was removed and replaced with medium containing SV40 large and small T antigen-helper-free viral supernatant in the presence of 4 mg/ml of polybrene (Sigma, St. Louis, Mo., USA) for 48 h. The cells were allowed to recover for 72 h followed by selection with 1 mg/ml puromycin (Sigma) for up to 15 days. The immortalized osteoblasts were cultured in a-MEM containing 10% FBS and 1% penicillin and streptomycin (P/S) and characterized following the protocols below. Cell proliferation was detected by BrdU incorporation assays following the manufacturer's directions (QIA58, Calbiochem, Gibbstown, N.J., USA). To induce differentiation, the immortalized osteoblasts were plated at a density of 2×10⁴ cells per well in a 12-well plate and 4×10⁴ cells per well in a 6-well plate and grown up to 21 days in a-MEM containing 10% FBS supplemented with 5 mM β-glycerophosphate and 25 g/ml ascorbic acid. ALP activity and Alizarin red-S histochemical staining for mineralization were performed as previously described (Xiao, Z., et al. (2006). Cilia-like structures and polycystin-1 in osteoblasts/osteocytes and associated abnormalities in skeletogenesis and Runx2 expression. J Biol Chem 281, 30884-30895). Total DNA content was measured with a PicoGreen® dsDNA quantitation reagent and kit (Molecular Probes, Eugene, Oreg., USA). Protein concentrations of the supernatant were determined with a Bio-Rad protein assay kit (Bio-Rad, Hercules, Calif., USA). For gene expression profiles, 1.0 μg of total RNA were isolated from primary osteoblasts cultured 3, 12, and 18 days in differentiation media. The cDNAs were generated using an iScript reverse transcription kit (Bio-Rad, Hercules, Calif., USA). PCR reactions contained 20 ηg template (cRNA or cDNA), 375 ηmol each forward and reverse primers, 1×SsoFast™ EvaGreen® supermix (Bio-Rad, Hercules, Calif., USA) in a total of 10 μl reaction volume. The Ct of tested gene product from the indicated genotype was normalized to the Ct for cyclophilin A as previously described (Xiao, Z., et al., (2008). Polycystin-1 regulates skeletogenesis through stimulation of the osteoblast-specific transcription factor RUNX₂-II. J Biol Chem 283, 12624-12634), (Xiao, Z., et al. (2006). Cilia-like structures and polycystin-1 in osteoblasts/osteocytes and associated abnormalities in skeletogenesis and Runx2 expression. J Biol Chem 281, 30884-30895)

Immunofluorescence:

The immortalized control and Kif3a-deficient osteoblasts were grown on collagen-coated 4-well chambers at 1×10⁵ cells per well and kept at confluence at least 3 days. At the end of the culture, the cells were washed three times with PBS, fixed with cold 4% paraformaldehyde/0.2% Triton for 10 min at room temperature, and washed with PBS three times. The cells were incubated for 30 min in 1% BSA before incubation with primary acetylated alpha-tubulin antibody (1:4000, T6793, Sigma Aldrich, St. Louis, Mo., USA) for 1 h at room temperature. After washing three times in PBS, cells were treated with secondary Texas Red-labeled anti-mouse IgG (1:400, 715-076-150, Jackson ImmunoResearch, West Grove, Pa., USA) in 1% BSA for 1 h at room temperature and washed three times in PBS before mounting with ProLong® Gold antifade reagent (Invitrogen, P36935). Nuclei were counterstained with DAPI blue. Photographs were taken under a microscope with magnification of 40× and 100× for counting the number and measuring the length of primary cilia in these osteoblasts as previously described, respectively (Xiao, Z, et al. (2006). Cilia-like structures and polycystin-1 in osteoblasts/osteocytes and associated abnormalities in skeletogenesis and Runx2 expression. J Biol Chem 281, 30884-30895)

Intracellular calcium ([Ca²⁺]ᵢ) measurements in vitro: We measured basal intracellular calcium ([Ca²⁺]ᵢ) concentration and flow-induced intracellular calcium response in immortalized control and Kif3a-deficient osteoblasts as previously described (Xiao, Z., Dallas, et al., (2011) Conditional deletion of Pkd1 in osteocytes disrupts skeletal mechanosensing in mice. FASEB J 25, 2418-2432). Briefly, the immortalized cells were cultured on type I rat tail collagen-coated 40-mm diameter glass slides at 80-90% confluence in alpha-minimal essential medium (a-MEM) containing 2% fetal bovine serum (FBS) and 1% P/S for 3 days. The cells were loaded with 3 μM Fura2-AM (Molecular Probes, Eugene, Oreg., USA), a fluorescent Ca²⁺ probe, in HBSS that contained 2% FBS and 20 mM HEPES for 30 min at 37° C. Loaded cells were incubated for an additional 45 min with HBSS alone to ensure complete deesterification of the fluorescent molecule. A glass slide was then placed in an FCS2 parallel plate flow chamber (Bioptechs, Inc., Butler, Pa., USA), 0.25×14×22 mm. A fresh bolus of flow media was added to the chamber, and the cells were left undisturbed for 30 min. The flow media consisted of phenol-free a-MEM and 2% FBS equilibrated with 5% CO₂/95% air at 37° C. The chamber was mounted on the stage of an inverted microscope with CCD camera to allow real-time record of fluorescence intensity (F340/F380 ratio) to generate ratiometric video images of individual static cells or cells exposed to pulsatile laminar fluid flow (Intracellular Imaging, Inc., Cincinnati, Ohio, USA). To obtain the optimal FFSS to induce response of intracellular calcium in individual osteoblasts, the immortalized control cells were exposed to various pulsatile laminar fluid flow rates resulting in shear stresses of 0.69, 1.56, 6.24, and 9.5 dynes/cm². To assess mechanoresponsive gene expression, total RNA was harvested and the cells subjected to FFSS (6.24 dynes/cm²) for 30 min and then returned to static culture for 30 min (post-FFSS) based on previous studies in osteoblasts (Mehrotra, M., et al., (2006). Role of Cbfa1/Runx2 in the fluid shear stress induction of COX-2 in osteoblasts. Biochem Biophys Res Commun 341, 1225-1230.)

The immortalized control and conditional Kif3a null osteoblasts were cultured in α-MEM containing 10% FBS and 1% P/S. To examine if conditional deletion of Kif3a (disruption of ciliogenesis) has an impact on Hh signaling in osteoblasts, a number of 1.5×10⁶ cells were transfected with 3.0 μg of Gli-responsive luciferase reporter construct (8×Gli-Luc) (Zhao, M., et al., (2009). Inhibition of microtubule assembly in osteoblasts stimulates bone morphogenetic protein 2 expression and bone formation through transcription factor Gli2. Mol Cell Biol 29, 1291-1305), 3.0 μg of pcDNA3.1 empty vector, and 0.6 μg of Renilla luciferase-null (RL-null) as internal control plasmid by electroporation using Cell Line Nucleofector Kit R according to the manufacturer's protocol (Amaxa, Inc., Gaithersburg, Md.). The cells were cultured in a-MEM supplemented with 1% FBS, and the relative luciferase activity of cell lysates was measured by a luciferase assay kit (Promega, Madison, Wis.) 72 h after transfection in the presence or absence of 1 μg/ml of recombinant mouse sonic hedgehog N-terminus (Shh-N) treatment for the last 8 h (Qiu, N., et al., (2010). Kif3a deficiency reverses the skeletal abnormalities in Pkd1 deficient mice by restoring the balance between osteogenesis and adipogenesis. PLoS One 5, e15240). We also isolated the total RNA for real-time RT-PCR analysis.

To explore potential abnormalities of the Wnt pathway in conditional Kif3a null mice, control (Kif3a^{flox/+}) and conditional Kif3a null (Kif3a^{Oc-cko}) osteoblasts were transiently cotransfected with 3.0 μg of Super 8×TOPFlash luciferase reporter plasmid (8×TOPFlash-Luc), 3.0 μg of pcDNA3.1 empty vector, or 0.6 μg of Renilla luciferase-null (RL-null, Promega, Madison, Wis.) as an internal control by electroporation. Promoter activity was assessed by measuring luciferase activity 48 h after transfection in the presence or absence of 100 ng/ml of recombinant Wnt3a treatment for the last 8 h. (Xiao, Z., et al., (2010). Conditional disruption of Pkd1 in osteoblasts results in osteopenia due to direct impairment of bone formation. J Biol Chem 285, 1177-1187). Total RNA was also isolated for real-time RT-PCR analysis.

To examine the amounts of cytoplasmic Cox-2, Gli-2, and β-catenin, the cells were prepared using 1× Passive Lysis Buffer for 30 minutes at 4° C. (Promega, Madison, Wis.) and centrifuged at 100,000 g for 45 minutes at 4° C. Protein concentrations of the supernatant were determined with a Bio-Rad protein assay kit (Bio-Rad, Hercules, Calif.). Equal quantities of protein were subjected to NuPAGE™ 4-12% Bis-Tris Gel (Invitrogen, Carlsbad, Calif.) and were analyzed with standard Western blot protocols (HRP-conjugated secondary antibodies from Santa Cruz Biotechnology and ECL from Amersham Biosciences, Buckinghamshire, UK). Antibody against Cox-2 (4842S) was from Cell Signaling Technology (Beverly, Mass.). Antibody against Gli-2 (ab7195, 80 kDa) was purchased from Abcam (San Francisco, Calif.). Anti-β-catenin (sc-7199) and Anti-β-actin (sc-47778) antibodies were from Santa Cruz Biotechnology (Santa Cruz, Calif.).

The differences between two groups were evaluated by unpaired t-test and multiple groups by one-way analysis of variance. All values are expressed as means±SD. All computations were performed using GraphPad Prism5 (GraphPad Software Inc. La Jolla, Calif., USA).

### Example 3

In the fibula fracture study, 12-week-old mice will be anesthetized with isoflurane as verified by toe pinch. The procedure follows that used previously in Dr. Hamrick's Lab (Kellum E, et al., (GDF-8) deficiency increases fracture callus size, Sox-5 expression, and callus bone volume. Bone. 2009 January; 44 (1):17-23).Briefly, the hair from the lateral side of the left hindleg will be shaved, and the leg cleaned with betadine and ethyl alcohol. A 5-7 mm incision will be made along the lateral side of the lower leg. For the fibular osteotomy, the midshaft of the fibula will be exposed by blunt dissection and a transverse fracture made in the fibula with microtenotomy scissors. The surgical wound will be closed using skin glue (3M Vetbond, Saint Paul Minn.) and the mouse receives an analgestic injection once. The mice will be housed for 4 weeks without disturb. Then the fibulas from different groups will be harvested for micro-CT 3D image analyses.

The principles, preferred embodiments and modes of operation of the present invention have been described in the foregoing specification. The invention which is intended to be protected herein, however, is not to be construed as limited to the particular forms disclosed, since these are to be regarded as illustrative rather than restrictive. Variations and changes may be made by those skilled in this art without departing from the spirit of the invention.

1. A method of detecting impaired mechanosensing function in bone cells in a subject comprising:
detecting the amount of one or more biomarker(s) in the mechanosensing complex in a biological sample of the subject, wherein the mechanosensing complex comprise polycystin-complex and primary cilia, and
calculating the amount of the one or more biomarker(s) in the sample and comparing to a control level of the biomarkers, wherein a measurable difference in the amount of the one or more biomarker(s) in the sample as compared to the control level indicates a greater likelihood that the subject suffers from impaired mechanosensing function.

2. The method of claim 1, wherein the polycystin-complex comprises Pkd1 and Pkd2.

3. The method of claim 1, further comprising detecting the amount of Pkd1 in a sample.

4. The method of claim 1, further comprising detecting the amount of Pkd2 in a sample.

5. The method of claim 1, wherein the primary cilia comprises Kif3a.

6. The method of claim 1, further comprising detecting the amount of Kif3a in a sample.

7. The method of claim 1, wherein the biological sample comprises blood, serum, plasma, bone tissues, femur tissues, osteoblasts and osteocytes.

8. The method of claim 2, wherein deletion of said Pkd1 impairs the activity of the mechanosensing complex and results in osteopenia in said subject.

9. The method of claim 2, wherein the deletion of said Pkd1 reduces bone formation and impairs mechanoresponsive gene expression in said subject.

10. The method of claim 2, wherein said Pkd1 in mature osteoblasts/osteocytes functions as a mechanosensor essential for bone cell responses to flow and mechanical loading in said subject.

11. The method of claim 5, wherein the deletion of said Kif3a disrupts primary cilia formation and function in said subject.

12. The method of claim 5, wherein the deletion of said Kif3a impairs osteoblast-mediated bone formation in said subject.

13. The method of claim 1, wherein the mechanosensing complex activity modulates bone formation and osteopenia in said subject.

14. The method of claim 1, wherein the technique to detect the amount of the biomarkers comprises RNA measuring assays and protein measuring assays.

15. The method of claim 14, wherein the RNA measuring assay comprises real time reverse transcription polymerase chain reaction.

16. The method of claim 14, wherein the protein measuring assays comprises western blot analysis and immunoassay analysis.

17. The method of claim 1 wherein said biomarker is measured by capturing the biomarker on an adsorbent surface of a probe and detecting the captured biomarkers by mass spectrometry.

18. A method of diagnosing impaired bone conditions in a subject by determining a measurable change in the amount of at least one biomarker in the mechanosensing complex, wherein the impaired bone condition comprises reduction in bone marrow density, bone volume and cortical thickness, and mineral apposition rate (MAR); reductions in osteoblast and osteoclastic markers; diminished osteoblast-mediated bone formation and osteoclast-mediated bone resorption; low-turnover osteopenia; postnatal bone formation; bone mass, structure, geometry, and mechanical properties; impaired osteoblastic differentiation and maturation; reduction in length of primary cilia; flow-induced intracellular calcium concentration; attenuated mechanoresponsive gene expression; impaired Hh signaling in osteoblasts; attenuated Wnt/p-catenin signaling in osteoblasts.

19. A method of treating bone loss and defective in bone formation in a subject in need thereof by modulating the activity of a mechanosensing complex in a subject, wherein the mechanosensing complex comprises polycystin-complex and primary cilia.

20. The method of claim 19 further comprising contacting the mechanosensing complex with a ligand which binds to polycystin-complex or primary cilia in a sufficient concentration to modulate the mechanosensing activity of the mechanosensing complex.

21. The method of claim 20, wherein the ligand is Triptolide.

22. The method of claim 21 further comprising monitoring the mechenosensing function in bone in a subject comprising:
detecting the amount of one or more biomarker(s) in the mechanosensing complex in a biological sample of a subject, wherein the mechanosensing complex comprise polycystin-complex and primary cilia, and
calculating the amount of the one or more biomarker(s) in the sample and comparing to a control level of the biomarkers, wherein a measurable difference in the amount of the one or more biomarker(s) in the sample as compared to the control level indicates a greater likelihood that the subject suffers from impaired mechanosensing function.

23. A kit to detect impaired mechanosensing function in bone comprising: agents capable of detecting the expression levels of at least one biomarker in a biological sample in the mechanosensing complex.

24. The kit of claim 23, wherein the agents comprises primers hybridizing to the expression products of at least one biomarker in the mechanosensing complex.

25. The kit of claim 23, wherein the primers are selected from the group consisting of SEQ ID 1-8.

26. The kit of claim 23, wherein the primers are selected from the group consisting of SEQ ID 47-52.

27. A kit to detect impaired mechanosensing function in bone comprising: a solid support having first agent to bind mechanosensing complex and, optionally, a second agent to identify the bound mechanosensing complex.

28. The kit of claim 25, wherein the first agent is a molecular probe or primer.

29. The kit of claim 25, wherein the second agent is a molecular probe or primer.


SEQUENCE LISTING (52 sequences)

SEQ ID NO: 1 - DNA, 25 bp, Mus musculus
cttctatcgccttcttgacgagttc

SEQ ID NO: 2 - DNA, 20 bp, Mus musculus
agggcttttcttgctggtct

SEQ ID NO: 3 - DNA, 22 bp, Mus musculus
tcgtgttcccttaccaaccctc

SEQ ID NO: 4 - DNA, 20 bp, Mus musculus
ctggtgacctatgtggtcat

SEQ ID NO: 5 - DNA, 20 bp, Mus musculus
ctggtgacctatgtggtcag

SEQ ID NO: 6 - DNA, 22 bp, Mus musculus
agccggtcttaacaagtatttc

SEQ ID NO: 7 - DNA, 20 bp, Mus musculus
atagggctcctggtgaacct

SEQ ID NO: 8 - DNA, 20 bp, Mus musculus
ccacagttgcactcaaatgg

SEQ ID NO: 9 - DNA, 20 bp, Mus musculus
gcctcacaaacaaccacaga

SEQ ID NO: 10 - DNA, 20 bp, Mus musculus
tcgctaacttgtggctgttg

SEQ ID NO: 11 - DNA, 19 bp, Mus musculus
tctggccttcctctctcag

SEQ ID NO: 12 - DNA, 20 bp, Mus musculus
agcaggagggcaataaggta

SEQ ID NO: 13 - DNA, 20 bp, Mus musculus
tctgatgagaccgtcactgc

SEQ ID NO: 14 - DNA, 23 bp, Mus musculus
gcctcagttgaataaacatgaaa

SEQ ID NO: 15 - DNA, 20 bp, Mus musculus
gttcctgcacagcttcacaa

SEQ ID NO: 16 - DNA, 20 bp, Mus musculus
gcagaaggaactgcaacaca

SEQ ID NO: 17 - DNA, 20 bp, Mus musculus
agttgacaggctccgagaaa

SEQ ID NO: 18 - DNA, 20 bp, Mus musculus
agtgaggaggacagcctgaa

SEQ ID NO: 19 - DNA, 22 bp, Mus musculus
cgcctgacaaacttttgagacc

SEQ ID NO: 20 - DNA, 20 bp, Mus musculus
aagccggtcaccgagttggt

SEQ ID NO: 21 - DNA, 20 bp, Mus musculus
aacaccacgagagtcctgct

SEQ ID NO: 22 - DNA, 20 bp, Mus musculus
agttgcccctactggaaggt

SEQ ID NO: 23 - DNA, 20 bp, Mus musculus
tggcttccacttcagctatg

SEQ ID NO: 24 - DNA, 24 bp, Mus musculus
gggtgcactggaccctgggtttac

SEQ ID NO: 25 - DNA, 20 bp, Mus musculus
caaggtgctccagaagatga

SEQ ID NO: 26 - DNA, 21 bp, Mus musculus
gcgtggaattcgatgaaatca

SEQ ID NO: 27 - DNA, 20 bp, Mus musculus
ctgcactgccaagactgaat

SEQ ID NO: 28 - DNA, 20 bp, Mus musculus
ttaaacgccagagccttctt

SEQ ID NO: 29 - DNA, 19 bp, Mus musculus
gctcacgtcgctcatcttg

SEQ ID NO: 30 - DNA, 19 bp, Mus musculus
ggatgaaatgcttgggaac

SEQ ID NO: 31 - DNA, 20 bp, Mus musculus
caagcagggttaagctcaca

SEQ ID NO: 32 - DNA, 20 bp, Mus musculus
cctcagtccataagccaagc

SEQ ID NO: 33 - DNA, 27 bp, Mus musculus
tcctcacccttcaattaaatcccacaa

SEQ ID NO: 34 - DNA, 20 bp, Mus musculus
aaacagcccagtgaccattc

SEQ ID NO: 35 - DNA, 20 bp, Mus musculus
tgatggtgaggtgtgcaaat

SEQ ID NO: 36 - DNA, 20 bp, Mus musculus
ggcactccacatcttggttt

SEQ ID NO: 37 - DNA, 20 bp, Mus musculus
gaggctctcgttggactcac

SEQ ID NO: 38 - DNA, 20 bp, Mus musculus
tgctccctgtttctgcttcc

SEQ ID NO: 39 - DNA, 20 bp, Mus musculus
gtgaggcgcttgcacttgca

SEQ ID NO: 40 - DNA, 20 bp, Mus musculus
gtaccagggcagagaagctg

SEQ ID NO: 41 - DNA, 20 bp, Mus musculus
gtggatagctcggtggtgtt

SEQ ID NO: 42 - DNA, 20 bp, Mus musculus
aggtaggcgatgctgttctt

SEQ ID NO: 43 - DNA, 24 bp, Mus musculus
cctggctcaccgccttggcttgtc

SEQ ID NO: 44 - DNA, 20 bp, Mus musculus
agtagctgcacgtgctctgt

SEQ ID NO: 45 - DNA, 20 bp, Mus musculus
cccgccatctagggttatga

SEQ ID NO: 46 - DNA, 20 bp, Mus musculus
ccacaatgttcatgccttct

SEQ ID NO: 47 - DNA, 19 bp, Mus musculus
agggcagacggaagggtgg

SEQ ID NO: 48 - DNA, 21 bp, Mus musculus
tctgtgagtttgtgaccagcc

SEQ ID NO: 49 - DNA, 19 bp, Mus musculus
agggcagacggaagggtgg

SEQ ID NO: 50 - DNA, 20 bp, Mus musculus
tggcaggtcaatggacgcag

SEQ ID NO: 51 - DNA, 20 bp, Mus musculus
gctatagacaggccgtcagc

SEQ ID NO: 52 - DNA, 20 bp, Mus musculus
gtctttggaggttcgttgga